(19) United States
(12) Patent Application Publication
(10) Pub. No.: US 2007/0208164 A1
(43) Pub. Date: Sep. 6, 2007

(54) Methods of synthesizing radiolabeled 3-cyano[14C]quinolines

(75) Inventors: John David Olszewski, Tappan, NY (US); Michael K. May, Nyack, NY (US); Dan Maarten Berger, New City, NY (US)

Correspondence Address: FITZPATRICK CELLA (WYETH), 30 ROCKEFELLER PLAZA, NEW YORK, NY, 10112-3800, US

(73) Assignee: WYETH, Madison, NJ (US)

(21) Appl. No.: 11/704,426

(22) Filed: Feb. 9, 2007

Related U.S. Application Data
(60) Provisional application No. 60/777,391, filed on Feb. 27, 2006.

(51) Int. Cl. C07F 5/00; C07D 403/14

(52) U.S. Cl. 534/11; 546/153; 544/263; 546/159

(57) ABSTRACT

The present invention is directed to radiolabeled 3-cyanoquinolines of the formula: 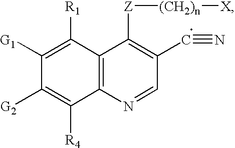
and methods of synthesizing the same, wherein G₁, G₂ R₁, R₄, Z, X, and n are as defined herein.
The present invention is also directed to a radiolabeled intermediate compounds of formula (VII): 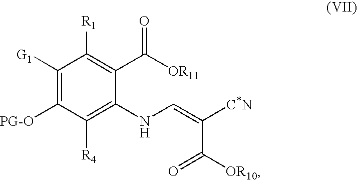
wherein PG, G₁, R₁, R₄, R₁₀, and R₁₁ are as defined herein, and synthesis of the same.

### CROSS-REFERENCE TO RELATED APPLICATIONS

This application claims the benefit of U.S. Provisional Application No. 60/777,391, filed Feb. 27, 2006, which is incorporated herein by reference.

### BACKGROUND OF THE INVENTION

1. Field of the Invention

This invention is directed to a method of synthesizing radiolabeled 3-[¹⁴C]cyanoquinolines. 3-cyanoquinoline derivatives are known to be potent chemo-agents.

2. Related Background Art

Protein kinases are a class of enzymes that catalyze the transfer of a phosphate group from ATP to a tyrosine, serine, threonine, or histidine residue located on a protein substrate, many of which play a role in normal cell growth. Correspondingly, several growth factor receptor proteins function as protein tyrosine kinases (PTKs) to effect signaling and are known as receptor tyrosine kinases (RTKs).

### BRIEF SUMMARY OF THE INVENTION

The present invention is directed to a method for preparing radiolabeled 3-cyanoquinolines comprising the steps of reacting:

a. a compound of formula (IIIa):

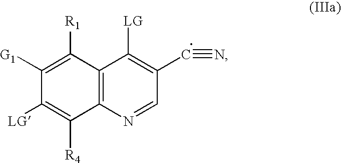

with a reagent of formula H-G₂ in the presence of a base to form the compound of formula (IIa):

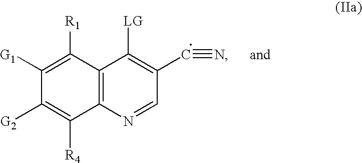

b. reacting the compound of formula (IIa) with a compound of formula H-Z-(CH₂)ₙ—X in the presence of a catalytic effective amount of an acid catalyst to produce a compound of formula (Ia):

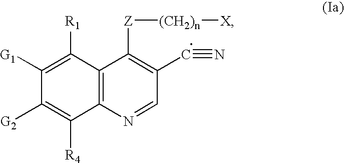

wherein

LG′ is —O-triflate or —O-mesylate,

X is a cycloalkyl of 3 to 7 carbon atoms, which may be optionally substituted with one or more alkyl of 1 to 6 carbon atoms, a pyridinyl, a pyrimidinyl, or a phenyl ring; wherein the pyridinyl, pyrimidinyl, or phenyl ring may be optionally mono-, di-, or tri-substituted with substituents selected from a group consisting of a halogen, an alkyl of 1-6 carbon atoms, an alkenyl of 2-6 carbon atoms, an alkynyl of 2-6 carbon atoms, azido, a hydroxyalkyl of 1-6 carbon atoms, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a benzoyl, an amino, an alkylamino of 1-6 carbon atoms, a dialkylamino of 2 to 12 carbon atoms, an alkanoylamino of 1-6 carbon atoms, an alkenoylamino of 3-8 carbon atoms, an alkynoylamino of 3-8 carbon atoms, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, a N,N-dialkylaminoalkyl of 3-10 carbon atoms, a N-alkylaminoalkoxy of 3-9 carbon atoms, a N,N-dialkylaminoalkoxy of 4-10 carbon atoms, mercapto, methylmercapto and benzoylamino, a bicyclic aryl or bicyclic heteroaryl ring system of 8 to 12 atoms where the bicyclic heteroaryl ring contains 1 to 4 heteroatoms selected from N, O, and S, wherein the bicyclic aryl or bicyclic heteroaryl ring may be optionally mono- di-, tri, or tetra-substituted with a substituent comprising a halogen, an oxo, a thiocarbonyl, an alkyl of 1-6 carbon atoms, an alkenyl of 2-6 carbon atoms, an alkynyl of 2-6 carbon atoms, azido, a hydroxyalkyl of 1-6 carbon atoms, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, a N,N-dialkylaminoalkyl of 3-carbon atoms, an N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, and a benzoylamino, a radical of the form:

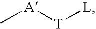

wherein

A′ is a pyridinyl, a pyrimidinyl, or a phenyl ring; wherein the pyridinyl, pyrimidinyl, or phenyl ring may be optionally mono- or di-substituted with a substituent comprising an alkyl of 1-6 carbon atoms, an alkenyl of 2-6 carbon atoms, an alkynyl of 2-6 carbon atoms, azido, a hydroxyalkyl of 1-6 carbon atoms, a halogen, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, a N-alkylaminoalkyl of 2-9 carbon atoms, a N,N-dialkylaminoalkyl of 3-10 carbon atoms, an N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, a alkenoyloxy of 3-8 carbon atoms, an alkynoyloxy of 3-8 carbon atoms, a carbamoyl, a N-alkylcarbamoyl of 2-7 carbon atoms, a N,N-dialkylcarbamoyl of 3-13 carbon atoms, and benzoylamino,

T is substituted at a carbon of the pyridinyl, pyrimidinyl, or phenyl ring with —NH(CH₂)ₘ—, —O(CH₂)ₘ—, —S(CH₂)ₘ—, —NR(CH₂)ₘ—, —(CH₂)ₘ—, —(CH₂)ₘNH—, —(CH₂)ₘO—, —(CH₂)ₘS—, —SO(CH₂)ₘ—, —SO₂(CH₂)ₘ—, —CO(CH₂)ₘ—, —(CH₂)ₘCO—, —(CH₂)ₘSO—, —(CH₂)ₘSO₂— or —(CH₂)ₘNR—,

L is an imidazole or a phenyl ring wherein the imidazole or phenyl ring are optionally substituted at a carbon or nitrogen with one, two, or three substituents comprising an alkyl of 1-6 carbon atoms, an alkenyl of 2-6 carbon atoms, an alkynyl of 2-6 carbon atoms, azido, a hydroxyalkyl of 1-6 carbon atoms, a halogen, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, an N,N-dialkylaminoalkyl of 3-10 carbon atoms, a N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, a benzoylamino, a 5- or 6-membered heteroaryl ring where the heteroaryl ring contains 1 to 3 heteroatoms selected from N, O, and S and where the heteroaryl ring may be optionally mono- or di-substituted with a substituent comprising a halogen, an oxo, a thiocarbonyl, an alkyl of 1-6 carbon atoms, an alkenyl of 2-6 carbon atoms, an alkynyl of 2-6 carbon atoms, azido, a hydroxyalkyl of 1-6 carbon atoms, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, a N,N-dialkylaminoalkyl of 3-carbon atoms, a N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, and a benzoylamino,

LG is selected from halo, O-triflate, and O-mesylate,

Z is —NH—, —O—, —S—, or —NR—,

R is alkyl of 1-6 carbon atoms,

G₂ is —O-alkyl, —O-phenyl, —O-benzyl, alkylamino of 1-6 carbon atoms, dialkylamino of 2 to 12 carbon atoms, phenylamino, benzylamino, heterocycloalkyl, heterocycloalkyl-heterocycloalkyl, where the heterocycloalkyl group or the heterocycloalkyl-heterocycloalkyl group contain a secondary amino functionality in the ring,

G₁, R₁, and R₄ are each, independently, a hydrogen, a halogen, a hydroxy, an amino, a hydroxyamino, a trifluoromethyl, a trifluoromethoxy, a mercapto, an alkyl of 1-6 carbon atoms, a cycloalkyl of 3-8 carbon atoms, an alkenyl of 2-6 carbon atoms, an alkynyl of 2-6 carbon atoms, an alkenyloxy of 2-6 carbon atoms, an alkynyloxy of 2-6 carbon atoms, a hydroxyalkyl of 1-6 carbon atoms, a mercaptoalkyl of 1-6 carbon atoms, a halomethyl, an alkoxymethyl of 2-7 carbon atoms, an alkoxy of 1-6 carbon atoms, a 2-methoxyethoxy, a 2-(2-methoxyethoxy)ethoxy, a cycloalkoxy of 3-8 carbon atoms, an alkylthio of 1-6 carbon atoms, a cycloalkylthio of 3-8 carbon atoms, an alkylsulfinyl of 1-6 carbon atoms, an alkylsulfonyl of 1-6 carbon atoms, an alkylsulfonamido of 1-6 carbon atoms, an alkenylsulfonamido of 2-6 carbon atoms, an alkynylsulfonamido of 2-6 carbon atoms, an alkylcarboxamido of 2-7 carbon atoms, a (N-alkyl)alkylcarboxamido of 3-13 carbon atoms, an alkenylcarboxamido of 3-7 carbon atoms, an (N-alkyl)alkenylcarboxamido of 4-13 carbon atoms, an alkynylcarboxamido of 3-7 carbon atoms, an (N-alkyl)alkynylcarboxamido of 4-13 carbon atoms, cyano, nitro, carboxy, a alkoxycarbonyl of 2-7 carbon atoms, an alkanoyl of 2-7 carbon atoms, an alkenoyl of 3-7 carbon atoms, a N-alkyl-N-alkenylamino of 4 to 12 carbon atoms, an N,N-dialkenylamino of 6-12 carbon atoms, phenylamino, benzylamino, phenoxy, phenyl, thiophenoxy, benzyl, an alkylamino of 1-6 carbon atoms, an alkanoyloxy of 2-7 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, a dialkylamino of 2 to 12 carbon atoms, an alkanoyloxymethyl group of 2-7 carbon atoms, an alkenoyloxymethyl group of 2-7 carbon atoms, an alkynoyloxymethyl group of 2-7 carbon atoms, azido, benzoyl, a carboxyalkyl of 2-7 carbons, and a carboalkoxyalkyl of 3-8 carbon atoms, —NR₆C(O)H; —N(C(O)R₆)C(O)R₆, —Y—(C(R₆)₂)ₚ-Het1, Y—(C(R₆)₂)ₚ-Het1-(C(R₆)₂)_{q}-Z¹-(C(R₆)₂)ᵣ-Het2,

Het1 is a 3-8 membered saturated heterocyclic ring containing one or more nitrogen, oxygen or sulfur atoms such as, but not limited to morpholine, thiomorpholine, thiomorpholine S-oxide, thiomorpholine S,S-dioxide, piperidine, pyrrolidine, aziridine, piperazine, tetrahydrothiophene, tetrahydrofuran, dioxane, 1,3-dioxolane pyrrole, tetrahydropyran, and diazepan; wherein Het1 is optionally mono- or di-substituted on a carbon or a nitrogen with R₆; optionally mono- or di-substituted on a carbon with hydroxy, —N(R₆)₂, or —OR₆; optionally mono or di-substituted on a carbon with the mono-valent radicals —(C(R₆)₂)ₛOR₆ or —[(C(R₆)₂)ₛN(R₆)₂]; or optionally mono or di-substituted on a saturated carbon with divalent radicals ═O or —O(C(R₆)₂)ₛO—,

Het2 is a heteroaryl selected from the group comprising morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane pyrrole, and tetrahydropyran; wherein Het2 is optionally mono- or di-substituted on a carbon or a nitrogen with R₆; optionally mono- or di-substituted on a carbon with hydroxy, —N(R₆)₂, or —OR₆; optionally mono or di-substituted on a carbon with the mono-valent radicals —(C(R₆)₂)ₛOR₆ or —[(C(R₆)₂)ₛN(R₆)₂]; or optionally mono or di-substituted on a saturated carbon with divalent radicals ═O or —O(C(R₆)₂)ₛO—,

R₆ is hydrogen, an alkyl of 1-6 carbon atoms, an alkenyl of 2-6 carbon atoms, an alkynyl of 2-6 carbon atoms, a cycloalkyl of 3-6 carbon atoms, an alkanoyl of 2-7 carbon atoms, a carbamoylalkyl of 2-7 carbon atoms, a hydroxyalkyl of 1-6 carbon atoms, a hydroxycycloalkyl of 3-6 carbon atoms, a carboxyalkyl of 2-7 carbon atoms, pyrrolidine, piperidine, or imidazole optionally substituted with methyl, a phenyl optionally mono-, di-, or tri-substituted with halogen, an alkoxy of 1-6 carbon atoms, trifluoromethyl, amino, an alkylamino of 1-3 carbon atoms, a dialkylamino of 2-6 carbon atoms, nitro, cyano, azido, halomethyl, an alkoxymethyl of 2-7 carbon atoms, an alkanoyloxymethyl of 2-7 carbon atoms, an alkylthio of 1-6 carbon atoms, hydroxy, a carboxyl, a alkoxycarbonyl of 2-7 carbon atoms, phenoxy, phenyl, thiophenoxy, benzoyl, benzyl, phenylamino, benzylamino, an alkanoylamino of 1-6 carbon atoms or alkyl of 1-6 carbon atoms,

Y is O, S, —NR₆C(O)—, —C(O)NR₆—, NR₆ or a bond,

Z¹ is O, S, —NR₆C(O)—, —C(O)NR₆—, NR₆ or a bond, p is 0-4, q is 0-4, r is 0-4, s is 1-6,

and at least one of the groups, R₁, R₂, R₃ or R₄ is consisting of —Y—(C(R₆)₂)ₚ-Het1-(C(R₆)₂)_{q}-Z-(C(R₆)₂)ᵣ-Het2,

m is 0-3, and

n is 0-1.

The invention is also directed to a method for preparing radiolabeled 3-cyanoquinolines comprising the step of reacting

(i) a compound of formula H-G₂′, and

(ii) a 3-cyanoquinoline intermediate having formula (IIb): 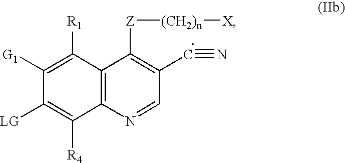
in the presence of a base to produce a compound of formula (Ib):

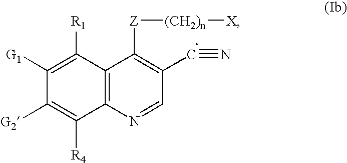

wherein:
G₂′ is Y′—R^{Y},

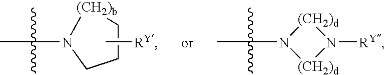

where b is 0, 1, or 2, and

d is 1 or 2,

Y′ is selected from —NR′—, —O— and —S—,

where R′ is alkyl of 1 to 6 carbon atoms,

R^{Y} is selected from alkyl, alkenyl, alkynyl, aryl, benzyl, heterocycloalkyl, -aryl-aryl, -aryl-heterocycloalkyl, -heterocycloalkyl-aryl, and heterocycloalkyl-heterocycloalkyl,

R^{Y′} is selected from alkyl, halo, haloalkyl, alkenyl, alkynyl, aryl, benzyl, heterocycloalkyl, alkoxy, haloalkoxy, aryloxy, —O—CH₂-phenyl, —O-heterocycloalkyl, dialkylamino, —N(alkyl)(aryl), diarylamino, —S-alkyl, —S-aryl, —S-heterocycloalkyl, and CN,

R^{Y″} is selected from alkyl, halo, haloalkyl, alkenyl, alkynyl, aryl, benzyl, and heterocycloalkyl, any of which may be substituted with suitable substituents, LG is selected from halo, —O-triflate and —O-mesylate, and

X, A′, T, L, Z, R, G₁, R₁, R₄, m, and n are as defined above.

Another aspect of the present invention is the method for preparing radiolabeled compounds of formula (VII): 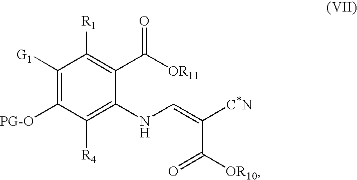
comprising the step of reacting a compound of formula R¹⁰O—C(O)CH₂—C*N and an intermediate having formula (VIII): 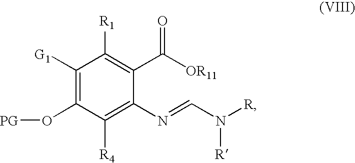
wherein PG is selected from benzyl, acetyl, methoxymethyl, benzyloxymethyl, methoxyethoxymethyl, 4-methoxybenzyl, and —C(O)O—CH₂—CCl₃.

R and R′ are independently alkyl, aryl or benzyl,

R¹⁰ is selected from alkyl, aryl, cycloalkyl and heterocycloalkyl,

R¹¹ is selected from alkyl, aryl, cycloalkyl and heterocycloalkyl, and

G₁ and R₁-R₄ are as defined above.

The present invention is also directed to a radiolabeled compound of formula (VII): 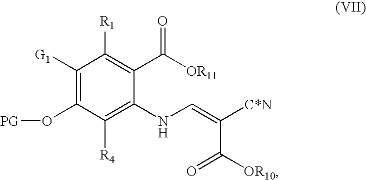
wherein PG, G₁, R₁, R₄, R₁₀, and R₁₁ are as defined above.

Yet another embodiment of this invention is a radiolabeled compound of formula (IIb): 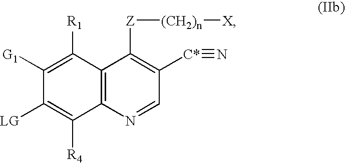
wherein LG is selected from halo, —O-triflate and —O-mesylate and

X, Z, G₁, R₁, R₄, and n are as defined above.

### DETAILED DESCRIPTION OF THE INVENTION

The radiolabeled 3-cyano[¹⁴C]quinolines of formulas (Ia) and (Ib) are carbon-14 isotope labeled derivatives and possess biological activity. Such radiolabeled molecules are useful because they allow for tracing the molecule in physiological processes occurring in living organisms. These compounds are [¹⁴C] labeled on the carbon of the 3-cyano moiety. This radiolabel may be introduced by reacting halogenated acetic acid with [¹⁴C]-potassium cyanide to form 2-cyano[¹⁴C]acetic acid, which then may be esterified and reacted with an substituted phenylamidine of formula (VIII) to give a radiolabeled compound of formula (VII). This compound then may be cyclized using 1,8-diazabicyclo(5.4.0)undec-7-ene (DBU) and triflic acid to form the 4-oxo-3-cyan[¹⁴C]-1,4-dihydroquinoline of formula (VI). This keto compound then may be halogenated to give a 3-cyano[¹⁴C]-quinoline of formula (V).

The 4-halo-3-cyano[¹⁴C]quinoline compound can then be deprotected at 7-position, in order to convert the 7-position functionality into a leaving group, and form a compound of formula (IIIb), which can then be reacted with a reagent of formula H-Z-(CH₂)ₙ—X to form a compound of formula (IIb). The 3-cyano[¹⁴C]quinoline compound of formula (IIb) can then be reacted with a reagent of formula H-G₂′, under basic conditions, to substitute the leaving group at the 7-position to make the final product, a compound of formula (Ib).

Alternatively, the 4-halo-3-cyano[¹⁴C]quinoline compound of formula (IIIa) can be reacted under basic condition with a nucleophilic reagent of formula H-G₂ to form a compound of formula (IIa). This compound is then arylated at the 4-position by reagent of formula H-Z-(CH₂)ₙ—X to form the final product, a compound of formula (Ia).

This method is a way of preparing a stable carbon-14 labeled isotope of the compounds disclosed herein.

For purposes of this invention the term “alkyl”, unless stated otherwise, includes both straight and branched alkyl moieties, which can contain as many as 12 carbon atoms. Preferably, the alkyl moiety contains between 1 to 6 carbon atoms, though 1 to 4 carbon atoms is more preferable.

For purposes of this invention, the term “alkenyl” refers to a radical aliphatic hydrocarbon containing one double bond and includes both straight and branched alkenyl moieties of 2 to 7 carbon atoms. Such alkenyl moieties may exist in the E or Z configurations; the compounds of this invention include both configurations.

For purposes of this invention, the term “alkynyl” includes both straight chain and branched moieties containing 2 to 6 carbon atoms having at least one triple bond.

For purposes of this invention, the term “cycloalkyl” refers to alicyclic hydrocarbon groups having 3 to 12 carbon atoms, but is preferably 3 to 7 carbon atoms, and includes but is not limited to: cyclopropyl, cyclobutyl, cyclopentyl, cyclohexyl, cycloheptyl, norbornyl, or adamantyl.

For purposes of this invention the term “aryl” is defined as an aromatic hydrocarbon moiety and may be substituted or unsubstituted. An aryl group preferably contains 6 to 12 carbon atoms and may be selected from, but not limited to, the group: phenyl, α-naphthyl, β-naphthyl, biphenyl, anthryl, tetrahydronaphthyl, phenanthryl, fluorenyl, indanyl, biphenylenyl, acenaphthenyl, acenaphthylenyl, or phenanthrenyl groups. An aryl group may be optionally mono-, di-, tri- or tetra-substituted with substituents selected from, but not limited to, the group consisting of alkyl, acyl, alkoxycarbonyl, alkoxy, alkoxyalkyl, alkoxyalkoxy, cyano, halogen, hydroxy, nitro, trifluoromethyl, trifluoromethoxy, trifluoropropyl, amino, alkylamino, dialkylamino, dialkylaminoalkyl, hydroxyalkyl, alkoxyalkyl, alkylthio, —SO₃H, —SO₂NH₂, —SO₂NHalkyl, —SO₂N(alkyl)₂, —CO₂H, CO₂NH₂, CO₂NHalkyl, and —CO₂N(alkyl)₂. Preferred substituents for aryl include: alkyl, halogen, amino, alkylamino, dialkylamino, trifluoromethyl, trifluoromethoxy, arylalkyl, and alkylaryl.

For purposes of this invention the term “heteroaryl” is defined as an aromatic heterocyclic ring system (monocyclic or bicyclic) where the heteroaryl moieties are five or six membered rings containing 1 to 4 heteroatoms selected from the group consisting of S, N, and O, and include but is not limited to: (1) furan, thiophene, indole, azaindole, oxazole, thiazole, isoxazole, isothiazole, imidazole, N-methylimidazole, pyridine, pyrimidine, pyrazine, pyrrole, N-methylpyrrole, pyrazole, N-methylpyrazole, 1,3,4-oxadiazole, 1,2,4-triazole, 1-methyl-1,2,4-triazole, 1H-tetrazole, 1-methyltetrazole, benzoxazole, benzothiazole, benzofuran, benzisoxazole, benzimidazole, N-methylbenzimidazole, azabenzimidazole, indazole, quinazoline, quinoline, pyrrolidinyl; (2) a bicyclic aromatic heterocycle where a phenyl, pyridine, pyrimidine or pyridizine ring is: (i) fused to a 6-membered aromatic (unsaturated) heterocyclic ring having at least one heteroatom; (ii) fused to a 5-membered aromatic or nonaromatic (unsaturated) heterocyclic ring having at least one heteroatom selected from O, N or S. Preferably a bicyclic heteroaryl group contains 8 to 12 carbon atoms. Preferred substituents for heteroaryl include: alkyl, halogen, amino, alkylamino, dialkylamino, trifluoromethyl, trifluoromethoxy, arylalkyl, and alkylaryl.

For the purposes of this invention the term “phenylamino” is defined as a C₆H₆—NH— radical.

For the purposes of this invention the term “benzylamino” is defined as a C₆H₆—CH₂—NH— radical.

For purposes of this invention the term “heterocycloalkyl” refers to a substituted or unsubstituted alicyclic ring system (monocyclic or bicyclic) wherein the heterocycloalkyl moieties are 3 to 12 membered rings containing 1 to 6 heteroatoms selected from the group consisting of S, N, and O. Preferably a heterocycloalkyl contains 2 to 11 carbon atoms. Examples of heterocycloalkyl rings are, but not limited to 1,4-dioxanyl, hexahydroazepinyl, piperazinyl, piperidinyl, pyrrolidinyl, morpholinyl, thiomorpholinyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydrotetrazolyl, dihydrothiadiazolyl, dihydrothiazolyl, dihydrothienyl, dihydrotriazolyl, dihydroazetidinyl, dihydro-1,4-dioxanyl, tetrahydrofuranyl, and tetrahydrothienyl.

For the purposes of this invention the term “heterocycloalkyl-heterocycloalkyl” is defined as a heterocycloalkyl moiety, as defined herein, bonded to heterocycloalkyl radical, for example pyrrolidinyl-piperidine.

For the purposes of this invention the term “alkoxy” is defined as C₁-C₆-alkyl-O—.

For the purposes of this invention the term “aryloxy” is defined as aryl-O—; wherein aryl is as defined above.

For the purposes of this invention the term “heteroaryloxy” is defined as heteroaryl-O—; wherein heteroaryl is as defined above.

For purposes of this invention the term “arylalkyl” is defined as aryl-C₁-C₆-alkyl-; arylalkyl moieties include benzyl, 1-phenylethyl, 2-phenylethyl, 3-phenylpropyl, 2-phenylpropyl and the like.

For purposes of this invention the term “alkanoyloxymethyl” is defined as —CH₂OC(O)R, wherein R is alkyl of 1 to 6 carbon atoms.

For purposes of this invention the term “alkylthio” is defined as C₁-C₆-alkyl-S.

For purposes of this invention “alkylthioalkyl” denotes an alkyl group as defined above that is further substituted with an alkylthio as defined above.

For the purposes of this invention “alkoxyalkyl” denotes an alkyl group as defined above that is further substituted with an alkoxy as defined above.

The terms “alkylamino” and “dialkylamino” refer to moieties with one or two alkyl groups, wherein the alkyl chain is 1 to 6 carbons and the groups may be the same or different. The terms “monoalkylaminoalkyl” and “dialkylaminoalkyl” refer to monoalkylamino and dialkylamino moieties with one or two alkyl groups (the same or different) bonded to the nitrogen atom which is attached to an alkyl group of 1 to 6 carbon atoms. Preferably a dialkylaminoalkyl moiety consists of 3 to 10 carbon atoms and a monoalkylaminoalkyl moiety consists of from 2 to 9 carbon atoms.

The terms “alkylaminoalkoxy” and “dialkylaminoalkoxy” refer to alkylamino and dialkylamino moieties with one or two alkyl groups (the same or different) bonded to the nitrogen atom which is attached to an alkoxy group of 1 to 6 carbon atoms. Preferably a dialkylaminoalkoxy moiety consists of 3 to 10 carbon atoms and an alkylaminoalkoxy moiety consist of from 2 to 9 carbon atoms.

For purposes of this invention the term “benzoylamino” is defined as a Ph-OC(O)NH— moiety.

For purposes of this invention the term “carboxy” is defined as a —COOH moiety.

For purposes of this invention the term “alkanoylamino” is defined as a —NH—COOR moiety, wherein R is alkyl of 1 to 6 carbon atoms.

For purposes of this invention the term “alkenoylamino” and “alkynoylamino” are defined as a —NH—COOR moiety, wherein R is alkenyl or alkynyl of 3 to 8 carbon atoms.

For purposes of this invention the term “carboalkoxy” is defined as —CO₂R, wherein R is alkyl of 1 to 6 carbon atoms.

For purposes of this invention the term “carboalkyl” is defined as —COR, wherein R is alkyl of 1 to 6 carbon atoms.

For purposes of this invention the term “carboxyalkyl” is defined as a HOOCR— moiety, wherein R is alkyl of 1 to 6 carbon atoms.

For purposes of this invention the term “carboalkoxyalkyl” is defined as a —R—CO₂—R′ moiety, wherein R and R′ are alkyl and together consist of from 2 to 7 carbon atoms.

For purposes of this invention the term “aminoalkyl” is defined as H₂N-alkyl, wherein the alkyl group consists of 1 to 5 carbon atoms.

“Azido” is a radical of the formula —N₃.

“Acyl” is a radical of the formula —(C═O)-alkyl or —(C═O)-perfluoroalkyl wherein the alkyl radical or perfluoroalkyl radical is 1 to 6 carbon atoms; preferred examples include but are not limited to, acetyl, propionyl, butyryl, trifluoroacetyl.

For purposes of this invention the term “alkylsulfinyl” is defined as a R′SO— radical, where R′ is an alkyl radical of 1 to 6 carbon atoms. Alkylsulfonyl is a R′SO₂— radical, where R′ is an alkyl radical of 1 to 6 carbon atoms. Alkylsulfonamido, alkenylsulfonamido, alkynylsulfonamido are R′SO₂NH— radicals, where R′ is an alkyl radical of 1 to 6 carbon atoms, an alkenyl radical of 2 to 6 carbon atoms, or an alkynyl radical of 2 to 6 carbon atoms, respectively.

The term “substituent” is used herein to refer to an atom radical, a functional group radical or a moiety radical that replaces a hydrogen radical on a molecule. Unless expressly stated otherwise, it should be assumed that any of the substituents may be optionally substituted with one or more groups selected from: alkyl, halogen, haloalkyl, hydroxyalkyl, nitro, amino, hydroxy, cyano, alkylamino, dialkylamino, alkoxy, haloalkoxy, alkoxyalkyl, alkoxyalkoxy, oxo, alkylthio, mercapto, haloalkylthio, aryl, aryloxy, arylthio, heteroaryl, heteroaryloxy, heteroarylthio, acyl, —CO₂-alkyl, —SO₃H, —SO₂NH₂, —SO₂NH-alkyl, —SO₂NH-(alkyl)₂, —CO₂H, —CO₂NH₂, —CO₂NH-alkyl and —CO₂N-(alkyl)₂.

For the purposes of this invention the term “substituted” refers to where a hydrogen radical on a molecule has been replaced by another atom radical, a functional group radical or a moiety radical; these radicals being generally referred to as “substituents.”

For the purposes of this invention the term “radiolabeled” refers to where a carbon atom of the molecule has been replaced with a radioactive isotope, for example carbon-14. Herein such radioactive atoms have been denoted with a “*”, for example, in the following formula: 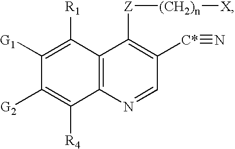
the carbon of the cyano group at the 3-position of the quinoline ring is indicated to be a radiolabeled carbon.

The term “protecting group” refers to a group introduced into a molecule to protect a sensitive functional group or specific position on the molecule from reacting when the molecule is exposed to reagents or conditions to transform or react another part of the molecule. Thereafter the protecting group can be removed. Suitable protecting groups are well known in the art and include acid-labile, base-labile, photoremovable, or removable under neutral conditions. See, e.g., Green, Protecting Groups in Organic Synthesis, Wiley 1991, 2^{nd} ed., pp. 309-405, which is incorporated herein by reference. The term “amine protecting group” refers to a moiety capable of protecting an amine functional group from reacting. Exemplary amine protecting groups for the present invention include acyl groups (such as acetyl), t-butoxycarbonyl (t-BOC), benzyloxycarbonyl, trifluoroacetyl, CH₃OC(O)—, EtOC(O)—, Fmoc, Troc, Phenoc, Teoc and PhC(O)— groups, and forming cyclicimides (e.g. phthalimide, maleimide, 2,3-dichloromaleimide, succinimide and dihydrophthalimide) and pyrroles (e.g. dimethylpyrrole). Exemplary phenolic protecting groups for the present invention include acyl groups (such as acetyl), benzyl, methoxymethyl (MOM), benzyloxymethyl (BOM), methoxyethoxymethyl (MEM), 4-methoxybenzyl, and —C(O)O—CH₂—CCl₃.

The compounds synthesized by this invention may contain an asymmetric carbon atom and may thus give rise to stereoisomers, such as enantiomers and diastereomers. The stereoisomers of the instant invention are named according to the Cahn-Ingold-Prelog System. While shown without respect to stereochemistry in formula (I), the present invention includes the synthesis of all the individual possible stereoisomers; as well as the racemic mixtures and other mixtures of R and S stereoisomers (scalemic mixtures which are mixtures of unequal amounts of enantiomers). It should be noted that stereoisomers of the invention having the same relative configuration at a chiral center may nevertheless have different R and S designations depending on the substitution at the indicated chiral center.

Another preferred embodiment of the method of the present invention synthesizing compounds of formula (Ia), is where G₂ is heterocycloalkyl-heterocycloalkyl. More preferable is wherein G₂ is 4-(pyrrolidin-1-yl)piperidin-1-yl.

Another preferred embodiment of the method of the present invention synthesizing compounds of formula (Ia), is where Z is NH, n is 0 and X is -A′-T-L.

Another preferred embodiment of the method of the present invention synthesizing compounds of formula (Ia), is where A′ is a phenyl ring. More preferable is where the phenyl ring is further substituted by halo, particularly Cl.

Another preferred embodiment of the method of the present invention synthesizing compounds of formula (Ia), is where T is —(CH₂)ₘS— and m is 0.

Another preferred embodiment of the method of the present invention synthesizing compounds of formula (Ia), is where L is 1-methyl-1H-imidazolyl.

Another preferred embodiment of the method of the present invention synthesizing compounds of formula (Ia), is where R₁ is H and/or R₄ is H.

Another preferred embodiment of the method of the present invention synthesizing compounds of formula (Ia), is wherein G₁ is alkyl, trifluoromethyl, alkoxy or trifluoromethoxy. More preferable is wherein G₁ is alkoxy, particularly methoxy.

Another preferred embodiment of the method of the present invention synthesizing compounds of formula (Ia), is where LG is halo, particularly where LG is Cl.

Another preferred embodiment of the method of the present invention synthesizing compounds of formula (Ia), is where the compound formed is 4-({3-chloro-4-[(1-methyl-1H-imidazol-2-yl)thio]phenyl}amino)-6-methoxy-7-(4-pyrrolidin-1-ylpiperidin-1-yl)quinoline-3-[¹⁴C]carbonitrile.

In a preferred embodiment of the method of the present invention the synthesis of compounds of formula (Ib), further comprises the step of reacting a compound of formula (IIIb): 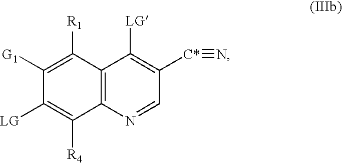
wherein LG′ is halo, more particularly Cl, with a reagent of formula H-Z-(CH₂)ₙ—X in an acidic environment to form a compound of formula (IIb). More preferable is where the method further comprises the step of reacting a compound of formula (IV): 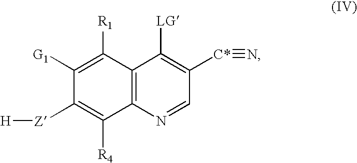
with a reagent effective to form a compound of formula (IIIb): 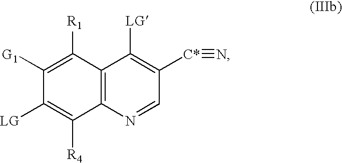
wherein Z′ is O or S, but O is the most preferred. Yet more preferable is where the method further comprises the step of deprotecting a compound of formula (V): 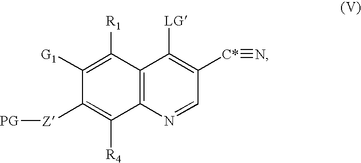
to form a compound of formula (IV): 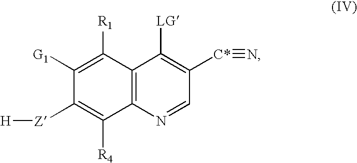
wherein PG is selected from benzyl, acetyl, methoxymethyl, benzyloxymethyl, methoxyethoxymethyl, 4-methoxybenzyl, and —C(O)O—CH₂—CCl₃, and is most preferably benzyl. Most preferred is where the method further comprises the steps of cyclizing a compound of formula (VIIb): 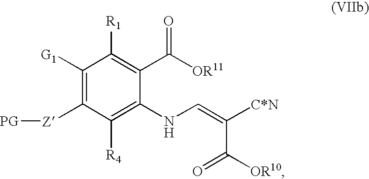
to form a compound of formula (VI): 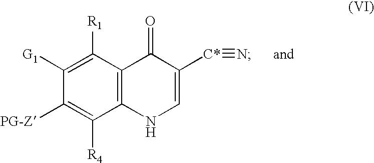
reacting the compound of formula (VI) with a reagent effective to form a compound of formula (V): 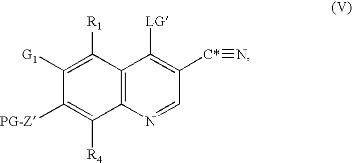
wherein R¹⁰ is selected from alkyl, aryl, cycloalkyl and heterocycloalkyl and R¹¹ is selected from alkyl, aryl, cycloalkyl and heterocycloalkyl, but R¹⁰ is most preferably alkyl, particularly t-butyl, and/or R¹¹ is most preferably alkyl, particularly methyl.

Another preferred embodiment of the method of the present invention synthesizing compounds of formula (Ib), is where G₂ is 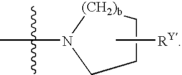
More preferably wherein b is 2 and RY′ is heterocycloalkyl.

Another preferred embodiment of the method of the present invention synthesizing compounds of formula (Ib), is where Z is NH, n is 0 and X is -A′-T-L. More preferable is wherein A′ is phenyl, and/or wherein T is —(CH₂)ₘS— and m is 0, and/or wherein L is 1-methyl-1H-imidazolyl. Most preferable is wherein in the phenyl ring is further substituted by halo, particularly Cl.

Another preferred embodiment of the method of the present invention synthesizing compounds of formula (Ib), is where R₁ is H and/or R₄ is H.

Another preferred embodiment of the method of the present invention synthesizing compounds of formula (Ib), is where G₁ is alkyl, trifluoromethyl, alkoxy or trifluoromethoxy, more preferably wherein G₁ is alkoxy, particularly methoxy.

Another preferred embodiment of the method of the present invention synthesizing compounds of formula (Ia), is where the compound formed is 4-({3-chloro-4-[(1-methyl-1H-imidazol-2-yl)thio]phenyl}amino)-6-methoxy-7-(4-pyrrolidin-1-ylpiperidin-1-yl)quinoline-3-[¹⁴C]carbonitrile.

Another preferred embodiment of the method of the present invention synthesizing compounds of formula (VII), is where R¹¹ is alkyl, more particularly methyl, and/or wherein R¹⁰ is alkyl, more particularly t-butyl.

Another preferred embodiment of the method of the present invention synthesizing compounds of formula (VII), is where R and R′ are alkyl, and more preferably are methyl.

Another preferred embodiment of the method of the present invention synthesizing compounds of formula (VII), is where G₁, R₁, and R₄ are each, independently, hydrogen, halogen, alkyl of 1-6 carbon atoms, hydroxymethyl, halomethyl, alkoxy of 1-6 carbon atoms, alkylthio of 1-6 carbon atoms, trifluoromethyl, trifluoromethoxy, cyano, nitro, phenoxy, phenyl, thiophenoxy, benzyl, alkylamino of 1-6 carbon atoms, or dialkylamino of 2 to 12 carbon atoms. More preferable is wherein G₁, R₁, and R₄ are each, independently, hydrogen, alkyl of 1-6 carbon atoms, trifluoromethyl, trifluoromethoxy or alkoxy of 1-6 carbon atoms. Yet more preferable is wherein R₁ is hydrogen, and/or wherein R₄ is hydrogen. Most preferable is wherein G₁ is alkoxy of 1-6 carbon atoms, particularly methoxy.

Another preferred embodiment of the method of the present invention synthesizing compounds of formula (VII), is where PG is benzyl.

Another preferred embodiment of the method of the present invention synthesizing compounds of formula (VII), is where the reaction occurs at about 25° C. or less.

Another preferred embodiment of the present invention of the compound of formula (VII), is where R¹¹ is alkyl, and more preferably methyl.

Another preferred embodiment of the present invention of the compound of formula (VII), is where R¹⁰ is alkyl, and more preferably t-butyl.

Another preferred embodiment of the present invention of the compound of formula (VII), is where G₁, R₁, and R₄ are each, independently, hydrogen, halogen, alkyl of 1-6 carbon atoms, hydroxymethyl, halomethyl, alkoxy of 1-6 carbon atoms, alkylthio of 1-6 carbon atoms, trifluoromethyl, trifluoromethoxy, cyano, nitro, phenoxy, phenyl, thiophenoxy, benzyl, alkylamino of 1-6 carbon atoms, or dialkylamino of 2 to 12 carbon atoms. More preferable is wherein G₁, R₁, and R₄ are each, independently, hydrogen, alkyl of 1-6 carbon atoms, trifluoromethyl, trifluoromethoxy or alkoxy of 1-6 carbon atoms. Yet more preferable is wherein R₁ is hydrogen, and/or wherein R₄ is hydrogen. Most preferably is wherein G₁ is alkoxy of 1-6 carbon atoms, particularly methoxy.

Another preferred embodiment of the present invention of the compound of formula (VII), is where PG is benzyl.

Another preferred embodiment of the present invention of the compound of formula (IIb), is where Z is NH, n is 0 and X is -A′-T-L. More preferable is wherein A′ is phenyl, and/or wherein T is —(CH₂)ₘS— and m is 0, and/or wherein L is 1-methyl-1H-imidazolyl. Most preferable is wherein in the phenyl ring is further substituted by halo, particularly Cl.

Another preferred embodiment of the present invention of the compound of formula (IIb), is wherein G₁, R₁, and R₄ are each, independently, hydrogen, halogen, alkyl of 1-6 carbon atoms, hydroxymethyl, halomethyl, alkoxy of 1-6 carbon atoms, alkylthio of 1-6 carbon atoms, trifluoromethyl, trifluoromethoxy, cyano, nitro, phenoxy, phenyl, thiophenoxy, benzyl, alkylamino of 1-6 carbon atoms, or dialkylamino of 2 to 12 carbon atoms. More preferable is wherein G₁ is hydrogen, alkyl of 1-6 carbon atoms, trifluoromethyl, trifluoromethoxy or alkoxy of 1-6 carbon atoms, and/or R₁ and R₄ are each, independently, hydrogen, alkyl of 1-6 carbon atoms, trifluoromethyl, trifluoromethoxy or alkoxy of 1-6 carbon atoms. Most preferred is where R₁ is H and/or R₄ is H, and/or G₁ is alkoxy of 1 to 6 carbon atoms, particularly wherein G₁ is methoxy.

Another preferred embodiment of the present invention of the compound of formula (IIb), is wherein LG is selected from —O-triflate and —O-mesylate, but most preferred is where LG is —O-triflate.
General Synthesis:

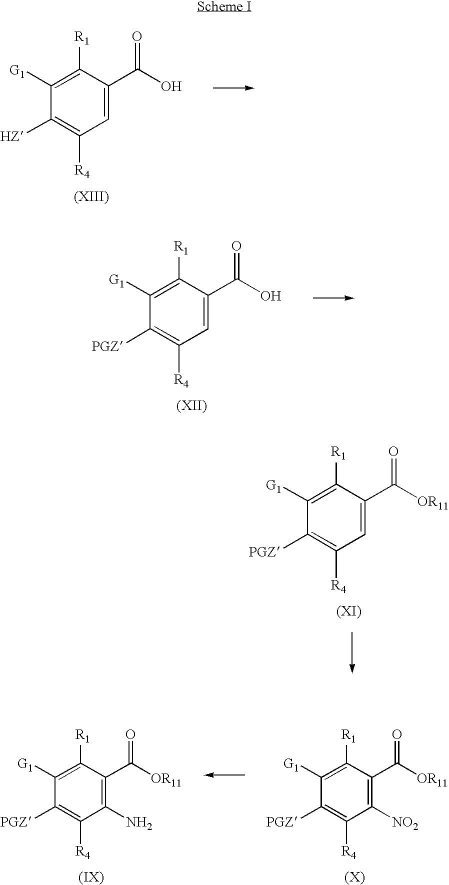

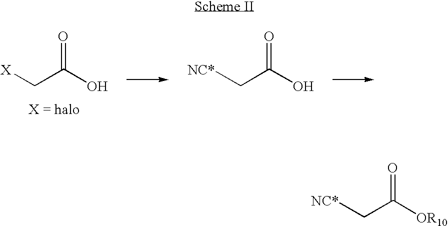

Scheme I illustrates synthesis of an intermediate compound of formula (IX), wherein G₁, R₁, R₄, Z′, PG and R₁₁ are as defined herein. The benzoic acid derivative of formula (XIII), wherein Z′ is either O or S, but more preferably is O, is protected with an appropriate protecting group to form a compound of formula (XII). One skilled in the art would know of such groups, as the literature is replete with examples of such groups, as well as how to form them and under what conditions such functionalities can be deprotected. When Z′ is O, the most preferred protecting group is benzyl.

The protected benzoic acid compound of formula (XII) is then esterified. Esterifications of benzoic acids are well documented in the literature and one skilled in the art would be aware of the many different conditions and reagents that could be used to effectuate this transformation under acidic or basic conditions. It is preferable that the ester be an alkyl ester, more preferably a methyl ester. Therefore, depending on the conditions needed based upon the substituents on the phenyl ring, an ester of formula (XI) could be formed by refluxing with an alkyl alcohol, such as methanol, with an acid, such as HCl or H₂SO₄. Alternatively, the ester can be formed under basic conditions by reacting the acid with a base, for example such as pyridine, triethylamine, K₂CO₃, Na₂CO₃ or NaH, and then introducing a haloalkyl reagent into the reaction, for example CH₃I. Other bases and reagents would be obvious to one skilled in the art.

The benzoate ester of formula (XI) is then nitrated to form a compound of formula (X). Nitration of an aryl rings is commonly known to those skilled in the art. One possible method involves reacting the ester with nitric acid and SnCl₄.

Then nitro benzoate ester of formula (X) is then reduced to the corresponding amino benzoate ester of formula (IX). This can be done using nickel chloride hexahydrate, but one skilled in art would be aware of other reagents and the necessary conditions to effectuate this reduction.

Scheme II shows the synthesis of a [¹⁴C] isotope labeled 2-cyano[¹⁴C]acetic ester, wherein R₁₀ is as defined herein, but is preferably alkyl and more preferably t-butyl.

First a halogenated acetic acid, for example such as 2-bromoacetic acid, is reacted with potassium[¹⁴C]cyanide to form 2-cyano[¹⁴C]acetic acid. This acid is then esterified. As stated herein, esterifications are well documented in the literature and a skilled artisan would be aware of a multitude of possible conditions and reagents to effectuate this conversion. One method would be to react the acid with an alkyl alcohol, for example such as t-butanol.

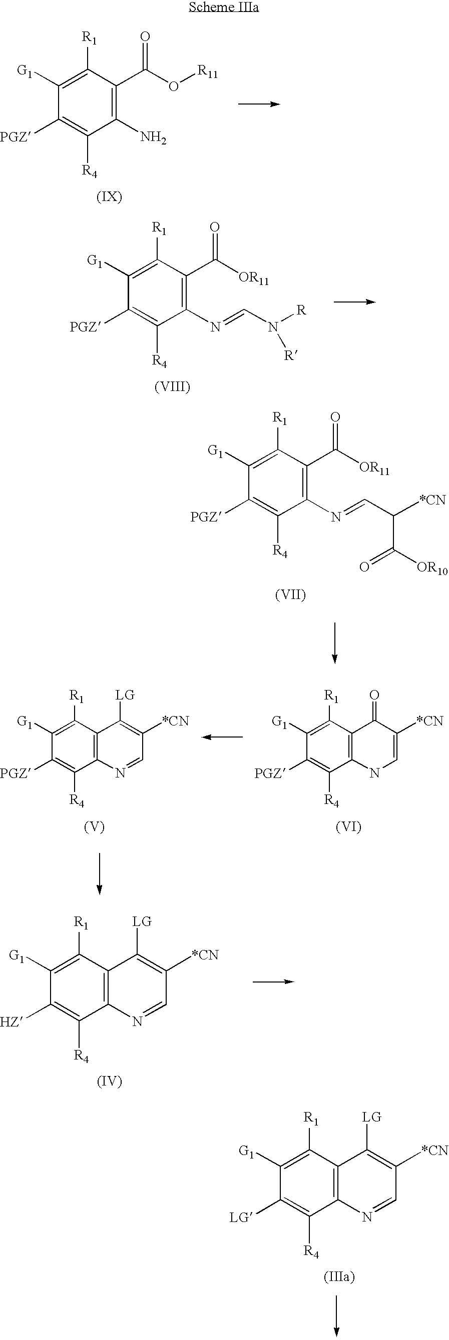

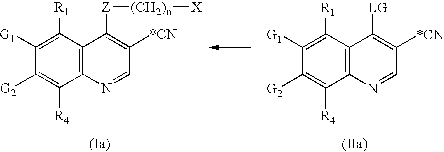

Scheme IIIa illustrates the synthetic pathway to the 3-cyano[¹⁴C]quinoline compounds of formula (Ia) from intermediates of formula (IX), wherein R, R′, G₁, G₂, R₁, R₄, R₁₀, R₁₁, Z, n, X, LG, LG′, Z′ and PG are defined herein.

The intermediate aminobenzoate ester of formula (IX) is converted to the corresponding amidine benzoate ester of formula (VIII) by reaction with DMF-DMA. This reaction is heated to at least 70° C. or greater, most preferably to about 83° C.

The amidine intermediate of formula (VIII) is then reacted with the 2-CN—[¹⁴C]-acetic ester, the preparation of which is shown in Scheme II, to form the radiolabeled intermediate of formula (VII).

The intermediate of formula (VII) is then cyclized to the 4-oxo-3-cyano[¹⁴C]-1,4-dihydroquinoline compound of formula (VI) by reacting the intermediate with triflic acid and DBU. It is preferable to perform its reaction at temperatures greater than about 80° C.

The 4-oxo-3-cyano[¹⁴C]-1,4-dihydroquinoline of formula (VI) is then reacted to form a 3-cyano[¹⁴C]quinoline of formula (V). The leaving group at the 4-position can be a halogen, —O-triflate or —O-mesylate, but is preferably chloro. This reaction can be performed by using a reagent of formula X′—C(O)—C(O)—X′, wherein X′ is a halogen, and is preferably oxalyl chloride. A skilled artisan would also know how to convert a halo group to either —O-triflate or —O-mesylate group.

The compound of formula (V) is then deprotected to give the compound of formula (IV). In a preferred embodiment Z′ is O and in this case the most preferred protecting group is benzyl, which can be deprotected by reaction with thioanisole in trifluoroacetic acid. Deprotection of a benzyl protected phenol is well known in the literature and one skilled in the art would know other reagents and conditions to effectuate this deprotection.

Once the deprotection is done, the HZ′-functional group can be converted to a leaving group, for example such as halo, —O-triflate or —O-mesylate, to form the compound of formula (IIIa). In the preferred embodiment where Z′ is O, it is preferable to form the corresponding —O-triflate by reacting the phenol with triflic anhydride under basic conditions. However, the corresponding —O-mesylate can also be readily formed, for example by reaction with mesylchloride. If desired, the corresponding halo compounds could also be formed by reacting the phenol with an appropriate reagent, for example such as POCl₃, PCl₅, phosgene/Ph₃P and Br₂/Ph₃P. One skilled in the art would be familiar with other reagents and conditions to form the corresponding 7-halo compound of formula (IIIa).

The intermediate of formula (IIIa) is then reacted with a reagent of formula H-G₂, wherein G₂ is defined herein, under basic conditions to form a compound of formula (IIa). The preferred bases for this reaction are pyridine, K₂CO₃, K₃PO₄, Na₂CO₃ and triethylamine, but one skilled in the art would be aware of other possible bases that could be used. The most preferred base is K₃PO₄. It is also preferable to add a catalytic amount of Pd₂(dba)₃ and di-t-butylphosphinobiphenyl. This reaction is preferably run at elevated temperatures, preferably about 85° C. Any solvent suitable for a Buchwald coupling can be used. One skilled in the art would be familiar with such solvents, but for reasons of solubility and stability, N-methylpyrrolidinone (NMP) is preferable.

The radiolabeled quinoline of formula (IIa) is then reacted with a reagent of H-Z-(CH₂)n-X to form the final product of formula (Ia). This can be performed with either a catalytic amount of acid, preferably triflic acid, or it can be performed under mildly basic conditions, preferably using pyridine HCl. One skilled would be aware of other reagents that could be used to facilitate this reaction. In a preferred embodiment Z is NH, n is 0 and X is a phenyl ring, wherein the phenyl ring may be further substituted by a halogen and/or —S-heteroaryl group. This reaction under the basic condition is preferably heated to temperatures greater than about 80° C., more preferably to about 110° C.

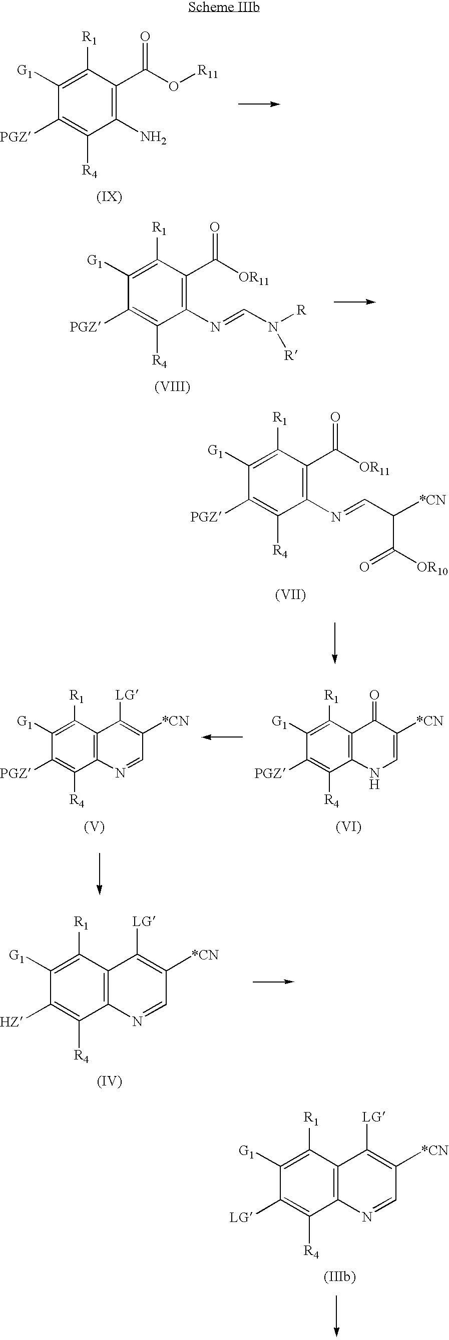

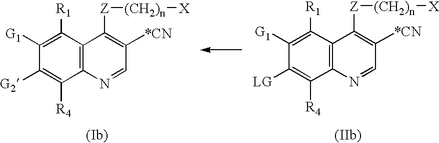

Scheme IIIb illustrates the synthetic pathway to the 3-cyano[¹⁴C]quinoline compounds of formula (Ib) from intermediates of formula (IX), wherein R, R′, G₁, G′₂, R₁, R₄, R₁₀, R₁₁, Z, n, X, LG, LG′, Z′ and PG are defined herein.

From the starting intermediate compound of formula (IX) to the radiolabeled intermediate of formula (IIIb), this general synthetic pathway is the same as that shown in Scheme IIIa. However, in Scheme IIIb, the radiolabeled 3-cyano[¹⁴C]quinoline intermediate of formula (IIIb) is reacted with a reagent of H-Z-(CH₂)n-X to form the final product of formula (IIb). This can be performed with either a catalytic amount of acid, preferably triflic acid, or it can be performed under mildly basic conditions, preferably using pyridine HCl. The basic conditions are the more preferable conditions. One skilled would be aware of other reagents that could be used to facilitate this reaction.

The compound of formula (IIb) is then reacted with a reagent of formula H-G₂′, wherein G₂′ is defined herein, under basic conditions to form the final product of formula (Ib). The preferred bases for this reaction are pyridine, K₂CO₃, K₃PO₄, Na₂CO₃ and triethylamine, but one skilled in the art would be aware of other possible bases that could be used. The most preferred base is K₃PO₄. It is also preferable to add a catalytic amount of Pd₂(dba)₃ and di-t-butylphosphinobiphenyl. This reaction is preferably run at elevated temperatures, preferably about 85° C. Any solvent suitable for a Buchwald coupling can be used. One skilled in the art would be familiar with such solvents. However, for reasons of solubility and stability, N-methylpyrrolidinone (NMP) is preferable. In a preferred embodiment Z is NH, n is 0 and X is a phenyl ring, wherein the phenyl ring may be further substituted by a halogen and/or —S-imidazole.
Specific Synthesis:

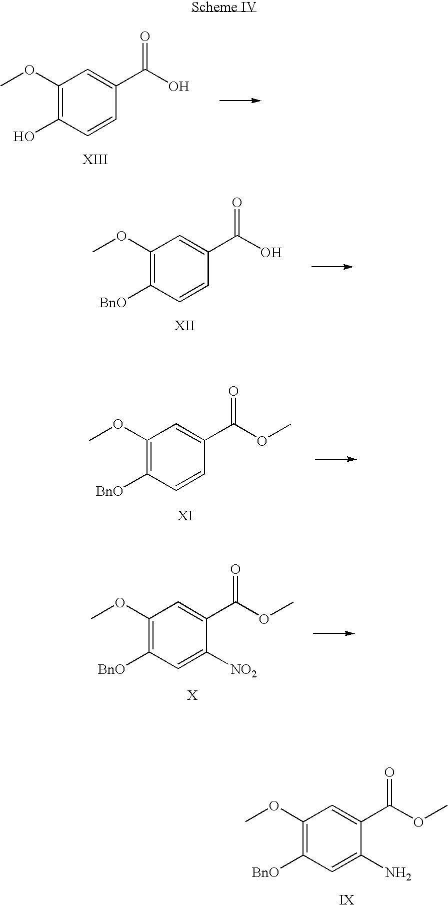

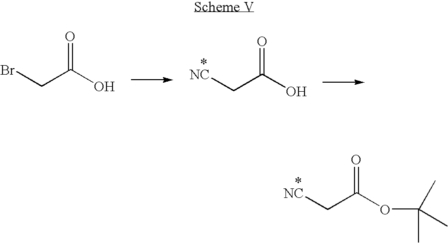

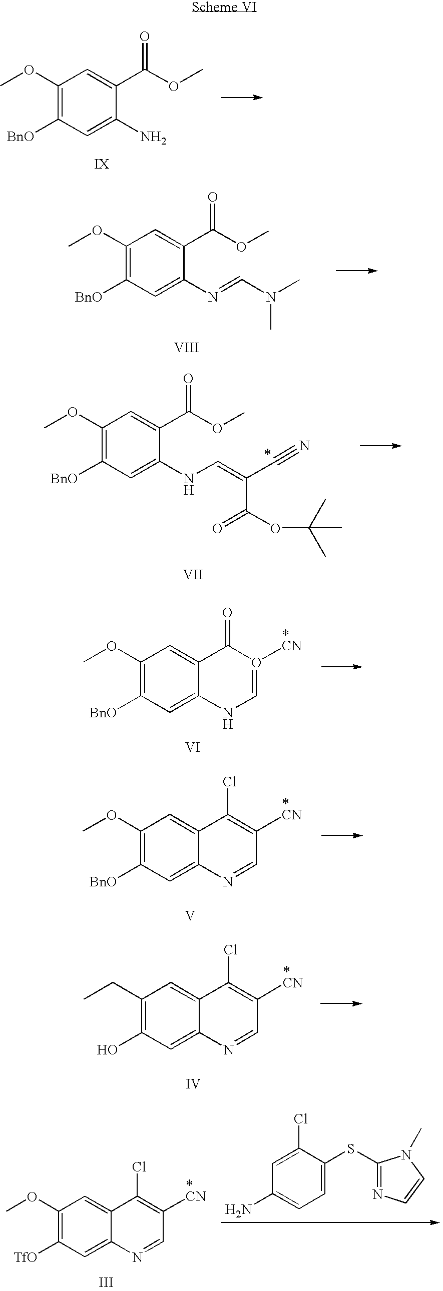

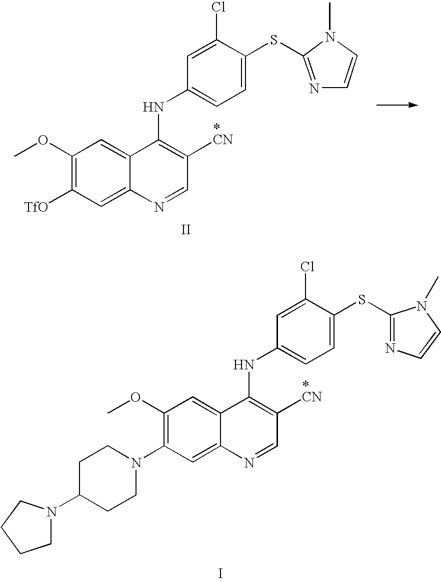

Scheme IV illustrates the specific synthesis of an intermediate aniline compound, 2-amino-4-benzyloxy-5-methoxy-methylbenzoate(IX), from the starting benzoic acid derivative (XIII).

First, the 4-hydroxyl group of the benzoic acid derivative (XIII) is protected by reacting it with benzyl chloride under basic conditions to form the corresponding benzyl ether(XII). Many bases can be utilized for this reaction and one skilled in the art would be familiar with them, but the preferred base is NaOH. Similarly, other benzyl halides, for example such as the corresponding iodo or bromo compounds, could be used in this reaction.

The protected benzoic acid compound (XII) is then esterified to form the corresponding methylbenzoate(XI). Esterifications of benzoic acids are well documented in the literature and one skilled in the art would be aware of the many different conditions and reagents that could be used to effectuate this transformation under acidic or basic conditions. It is preferable that the ester be formed under basic conditions by reacting the acid with a base, for example such as pyridine, triethylamine, K₂CO₃, Na₂CO₃ or NaH, and then introducing a halomethyl reagent into the reaction, for example CH₃I. The preferred base is K₂CO₃. It is also preferable that this reaction be heated, preferably to about 55° C.

The benzoate ester (XI) is then nitrated to form compound (X) by reacting the ester with nitric acid and SnCl₄. Nitration of an aryl ring is commonly known to those skilled in the art and other reagents and conditions would be known.

Then nitro benzoate ester (X) is then reduced to the corresponding aniline compound (IX). This can be done using nickel chloride hexahydrate and sodium borohydrate, but one skilled in art would be aware of other reagents and the necessary conditions to effectuate this reduction.

Scheme V shows the synthesis of a [¹⁴C] isotope labeled t-butyl-2-cyano[¹⁴C]acetate, from bromoacetic acid.

Bromoacetic acid is reacted with potassium[¹⁴C]cyanide to form 2-cyano[¹⁴C]acetic acid. This can be done at reflux temperature in aqueous solution. This acid is then esterified by reacting it with t-butanol in the presence of DCC at room temperature.

Scheme VI illustrates specific synthesis of 4-({3-chloro-4-[(1-methyl-1H-imidazol-2-yl)thio]phenyl}amino)-6-methoxy-7-(4-pyrrolidin-1-ylpiperidin-1-yl)quinoline-3-[¹⁴C]carbonitrile(I) from the intermediate aniline compound (IX), the preparation of which is shown in Scheme IV.

The protected aniline (IX) is reacted with DMF-DMA to form the amidine compound (VIII). This reaction is heated to at least 70° C. or greater, most preferably to about 83° C.

The amidine compound (VIII) was then reacted with t-butyl-2-cyano[¹⁴C]acetate(IX), the preparation of which is shown in Scheme V, to form (Z)-methyl 4-(benzyloxy)-2-(3-tert-butoxy-2-cyano-[¹⁴C]-3-oxoprop-1-phenylamino)-5-methoxybenzoate(VII). This reaction can be performed at room temperature using a protic solvent, such as isopropanol. This product can then be cyclized to form the 4-oxo3-cyano[¹⁴C]-1,4-dihydroquinoline compound (VI) using triflic acid and DBU with heating, preferably to temperatures greater than about 80° C.

The 4-oxo-3-cyano[¹⁴C]-1,4-dihydroquinoline compound (VI) is then reacted with oxalyl chloride to form the protected 4-chloro-6-methoxyquinoline-3-carbonitrile-[¹⁴C] (V). This reaction is preferably heated to temperatures greater than about 50° C., more preferably to about 65° C. This compound was then deprotected using trifluoroacetic acid and thioanisole to give the corresponding radiolabeled 7-hydroxyquinoline compound (IV), which is then readily converted to corresponding 7-triflate-quinoline compound (III) by reaction with triflic anhydride under basic conditions. One skilled in the art would know of appropriate bases to use in this reaction, for example, tertiary amine bases, such as triethylamine and pyridine. Pyridine is the preferred base.

4-chloro-3-cyano-[¹⁴C]-6-methoxyquinolin-7-yl trifluoromethanesulfonate(III) was then reacted with 3-chloro-4-(1-methyl-1H-imidazol-2-ylthio)aniline using pyridine hydrochloride to form 4-(3-chloro-4-(1-methyl-1H-imidazol-2-ylthio)phenylamino)-3-cyano-[¹⁴C]-6-methoxyquinolin-7-yl trifluoromethanesulfonate(II). This reaction is preferably heated to temperatures greater than about 80° C., more preferably to about 110° C.

The triflate compound (II) is then reacted with pyrrolidinyl-piperidine in the presence of K₃PO₄ (208 mg), Pd₂(dba)₃ (37 mg) and di-t-butylphosphinobiphenyl to form the final product (I), 4-({3-chloro-4-[(1-methyl-1H-imidazol-2-yl)thio]phenyl}amino)-6-methoxy-7-(4-pyrrolidin-1-ylpiperidin-1-yl)quinoline-3-[¹⁴C]carbonitrile. Any solvent suitable for a Buchwald coupling can be used. One skilled in the art would be familiar with such solvents. However, for reasons of solubility and stability, N-methylpyrrolidinone (NMP) is preferable.

### EXAMPLES

### Preparation of 4-benzyloxy-3-methoxy-benzoic acid

5.0 grams of 4-hydroxy-3-methoxy-benzoic acid and 15 mL of THF were combined at room temperature. 37.5 mL of 2 N NaOH was added and the mixture cooled on ice. 4.0 mL of benzyl chloride was diluted with THF and added to the reaction mixture in a dropwise manner over 30 minutes. After the addition was complete, the mixture was warmed to room temperature and then refluxed. After a total of 30 hours of reflux, the reaction mixture was cooled to room temperature and washed twice with hexane. The aqueous THF phase was concentrated in vacuo such that most of the THF was removed and a solid remained. 50 mL of 1 N NaOH was added to give a slurry. Concentrated HCl was added slowly until all the material dissolved and the product then precipitated out of the acidic solution. After the pH was adjusted to 1, a white precipitate was filtered off and washed with 10% HCl. The precipitate was dissolved in hot ethyl acetate, separately, dried over sodium sulfate, filtered and the filtrate evaporated. The resulting solid was recrystallized from hot ethyl acetate. The yield of the first crop was 3.34 grams. Additional material could be isolated from the filtrate. Proton NMR was in agreement with the proposed structure. (lit. ref. Synthesis 1990 pp. 81-84 by Thurston et. al.)

### Preparation of 4-benzyloxy-3-methoxy-methylbenzoate

2.84 g of 4-benzyloxy-3-methoxy-benzoic acid was combined with 3.04 g of potassium carbonate and 1.03 mL of iodomethane in 100 mL of acetone. The mixture was refluxed for 16 hours, cooled to room temperature and diluted with dichloromethane. The slurry was filtered and washed with dichloromethane and the filtrate concentrated to dryness. The solids were redissolved in dichloromethane, washed twice with water, dried over sodium sulfate and filtered. The filtrate was concentrated to a clear, colorless oil which was triturated with methanol to give a white solid in 90% yield (2.7 grams). Proton

NMR was in agreement with the proposed structure. (lit. ref. Organic Letters 1999 vol 1(11) pp 1835-37)

### Preparation of 2-nitro-4-benzyloxy-5-methoxy-methylbenzoate

1.15 g of 4-benzyloxy-3-methoxy-methylbenzoate was dissolved in 30 mL anhydrous dichloromethane under a nitrogen atmosphere. The mixture was cooled in a slush bath consisting of carbon tetrachloride and dry ice (−25 C). 6.83 mL of tin(IV)chloride (1.0 M in CH₂Cl₂) was added to an addition funnel followed by 368 μL of 90% nitric acid. This solution was dripped into the reaction mixture over 5 minutes. The resulting reaction mixture was stirred for 15 minutes in the slush bath, an equal volume of water added and the mixture warmed to room temperature. The resulting layers were separated and the aqueous phase extracted two times with ethyl acetate. The organic phases were combined, dried over sodium sulfate, filtered and concentrated to dryness. The residue was triturated with methanol to give the product as a pale yellow solid in 88% yield. Proton NMR was consistent with the proposed structure. (lit. ref. Organic Letters 1999 vol 1(11) pp 1835-37)

### Preparation of 2-amino-4-benzyloxy-5-methoxy-methylbenzoate

6.5 g of 2-nitro-4-benzyloxy-5-methoxy-methylbenzoate was dissolved in 2:1 CH₂Cl₂/MeOH (138 mL). 1.56 g of nickel chloride hexahydrate was added and the resulting green solution stirred until all solids dissolved. The reaction mixture was then cooled on ice and 2.56 g of sodium borohydride added slowly in 4 portions, with each portion being added at 7-8 minute intervals. After the last addition was complete, all the solvent was removed to result in a gray solid. 150 mL of cold 10% HCl was added and the mixture washed with a 100 mL rinse of 10% HCl. The aqueous phase extracted three more times with ethyl acetate. The organic phases were combined and dried over sodium sulfate. After filtration, the filtrate was concentrated to a pink-orange solid, which was recrystallized from methanol. The final product (2.13 grams) was isolated as a pale orange-brown solid. Proton NMR was consistent with the proposed structure. (lit. ref. Organic Letters 1999 vol 1(11) pp 1835-37).

### Preparation of (E)-methyl 4-(benzyloxy)-2-((dimethylamino)methyleneamino)-5-methoxybenzoate

2.39 g of 2-amino-4-benzyloxy-5-methoxy-methyl-benzoate was combined with 75 mL of isopropanol under a nitrogen atmosphere. 2.65 mL of DMF-DMA was added and the mixture refluxed for 4.5 hours. HPLC check at this time showed that 8% starting material remained. An additional 1 mL of DMF-DMA was added and reflux continued for 2 hours. HPLC check indicated less than 3% starting material remained. LCMS also indicated that a product of the desired molecular weight had been formed. The reaction mixture was cooled slowly to room temperature and stirred over night. A few seed crystals of authentic product were added to the stirring reaction mixture and the resulting slurry stirred for 30 minutes at room temperature. The off white solids were filtered off and washed with isopropanol. After drying under high vacuum at 30 C the yield was determined to be 88.9% (2.53 g).

### Preparation of cyano[¹⁴C]acetic acid

1.263 g of bromoacetic acid was combined with 30 mL water and stirred until all dissolved. 1.256 g of potassium carbonate was added in portions at room temperature while stirring. [¹⁴C]-potassium cyanide (500 mCi) was dissolved in minimal water and added to the reaction mixture followed by several water rinses (total volume of water was 15 mL). The reaction mixture was refluxed for 2 hours and cooled to room temperature. MS analysis showed that no starting material remained. After cooling the reaction mixture on ice, concentrated HCl was added dropwise with stirring until the solution was strongly acidic. Solid sodium chloride was added until the solution was saturated. The resulting mixture was extracted 6 times with ethyl acetate. The organic phases were combined, dried over MgSO4, filtered and concentrated to an orange oil that solidified under vacuum. The solid was dried for one hour to result in a 97.8% yield (774.6 mg, 489 mCi). (Procedure was based on the following reference: J. Lab. Compounds (1981) 28(8), p. 1107).

### Preparation of t-butyl-cyano-[¹⁴C]-acetate

489 mCi of cyano-[¹⁴C]-acetic acid was dissolved in 10 mL of anhydrous acetonitrile under argon and 1.1 mL of t-butanol added. 2.39 g of DCC was dissolved in 8 mL of acetonitrile and added slowly to the stirred reaction mixture. This was followed by a 4 mL rinse. After stirring for 2 hours at room temperature, another 0.5 eq of t-butanol was added followed by 0.5 eq DCC in 4 mL acetonitrile. The mixture was stirred for another hour at room temperature, filtered and the solids washed well with acetonitrile. The filtrate was concentrated to a green oil and the product was purified by silica gel chromatography using 6:1 hexane/ethyl acetate. The product was isolated as a yellow liquid in 66.6% yield (837.2 mg). RadioTLC and visualization of the product on TLC plates with iodine vapor was done next to a non-radioactive reference standard to isolate the desired material. RHPLC showed the material to be of >90% RCP. (based on Lit. procedure—J. Org. Chem. (2002) vol. 67 pp. 8975-82).

### Preparation of (Z)-methyl 4-(benzyloxy)-2-(3-tert-butoxy-2-cyano-[¹⁴C]-3-oxoprop-1-(phenylamino)-5-methoxybenzoate

841 mg of amidine was combined with 4 mL of isopropanol and stirred under argon at room temperature. The t-butyl-cyano-[¹⁴C]-acetate (326 mCi) was dissolved in 4 mL of IPA and added to the reaction mixture. This was followed by 4×4 mL rinses of IPA to give a total reaction volume of approximately 25 mL. The slurry was stirred at room temperature under argon for 68 hours. At this time, TLC and LCMS showed that no starting material remained. The slurry was filtered and the resulting solids washed with IPA. The solids were dried under vacuum to give a 77% yield based on starting amidine (125 mCi).

The filtrate contained t-butyl-cyano-[¹⁴C]-acetate, which had been used in excess in the reaction. Therefore the filtrate was concentrated and recycled via the same procedure (although using smaller volumes) to give additional material. Recycling was done until less than 20 mCi of t-butyl-cyano-[¹⁴C]-acetate remained. The final yield was 1.89 grams (236 mCi) or 72% based on t-butyl-cyano-[¹⁴C]-acetate.

### Preparation of 7-(benzyloxy)-6-methoxy-4-oxo-1,4-dihydroquinoline-3-carbonitrile-[¹⁴C]

The starting material (10, 295 mCi, 2.36 g) was combined with 80 mL of acetonitrile to result in a cream colored slurry. 95 μL of triflic acid was added in a dropwise manner while stirring. The reaction mixture was warmed to 80° C. and stirred for 30 minutes. 3.21 mL of DBU was added in a dropwise manner and the reaction mixture warmed to reflux. After 18 hours at reflux, LCMS indicated the desired product had been formed but starting material still remained. 2.2 mL of DBU was added and the mixture refluxed for another 8 hours but the reaction was still incomplete. 6 mL DBU was added and reflux continued for 16 hours. HPLC showed the reaction to be complete. Cooled to room temperature and removed the acetonitrile in vacuo. The resulting solution was added dropwise to a room temperature solution of 250 mL water and 6 mL concentrated HCl to result in an orange slurry. After all the material had been added, 100 mL of 10% HCl was added to ensure acidity. Another 5 ml acetonitrile was used to rinse the flask and added. The slurry was stirred for 2 hours at room temperature and the solids filtered off. The solids were washed with water and diethyl ether and dried under vacuum at room temperature for 72 hours. This resulted in an orange powder (235.5 mCi) in 80% yield. RHPLC shows RCP and CP to be approximately 80%.

### Preparation of 7-(benzyloxy)-4-chloro-6-methoxyquinoline-3-carbonitrile-[¹⁴C]

The following procedure was repeated 4 times with all reactions performed at approximately the same scale with the amount of reagent and solvent adjusted accordingly. 311 mg of ¹⁴C-cyanoquinoline was combined with 45 mL of anhydrous chloroform and 100 μL of anhydrous N,N-dimethylformamide. 176 μL of neat oxalyl chloride was added and the mixture refluxed for 30 minutes at which point HPLC was used to determine that the reaction was complete. After cooling to room temperature, 2 mL of methanol was added, the solvent removed and the product was purified by rapid filtration through a pad of silica gel (approx. 3″ tall and 2″ wide). Three 500 mL washes of dichloromethane were used to elute the product. Pure fractions were pooled and the solvent removed in vacuo to result in a yellow-orange solid. Impure fractions were pooled, concentrated and rechromatographed in the same manner. The product was obtained in 72% yield (1.04 grams, 175 mCi). It was characterized by LCMS and R-HPLC which showed 96.1% RCP and 91.5% CP.

### 4-chloro-7-hydroxy-6-methoxyquinoline-3-carbonitrile-[¹⁴C]

1.04 g of ¹⁴C-chlorocyanoquinoline (175 mCi) was dissolved in 100 mL of trifluoroacetic acid at room temperature under argon. 3.74 mL of thioanisole was added with stirring and the reaction mixture refluxed for 3 hours. HPLC showed that no starting material remained and the reaction was cooled to room temperature. The solvent was removed in vacuo to result in an orange oil. To this was added 100 mL of ice water and a stir bar. The flask was cooled on ice and the yellow precipitate stirred vigorously. Concentrated NH₄OH was added in a dropwise manner until pH 9-10 was obtained. The yellow solid was filtered off and washed with water (2×30 mL) and diethylether (1×30 mL). The solids were dried under vacuum over night. The filtrates were combined and stirred with 50 mL of CH₂Cl₂ at 45° C. for 10 minutes and the layers separated. This was repeated twice. The organic phases were combined, dried over sodium sulfate, filtered and the solvent removed. The resulting solid was combined with the original filtered solids, dissolved/suspended in minimal CH₂Cl₂ containing 1% methanol and loaded onto a pad of silica gel (8.5″ tall and 2″ wide). Additional warm solvent of the same composition was added to the top of the column to get all the material dissolved and loaded onto the silica gel. The column was rapidly eluted (approx. 12 mL/min) with the following: 500 mL CH₂Cl₂, 500 mL 1% methanol in CH₂Cl₂, 1000 mL of 2% methanol in CH₂Cl₂, and 5% methanol in CH₂Cl₂ until no more product eluted. TLC analysis of the eluate was performed with 95:5 CH₂Cl₂/MeOH. Pure fractions were combined and the solvent removed in vacuo to result in a bright yellow solid which was dried under vacuum to give a 79% yield of the desired product (595 mg, 138 mCi). HPLC and LCMS were used to characterize the product which was found to be >95% pure and had the correct parent ion.

### Preparation of 4-chloro-3-cyano-[¹⁴C]methoxyquinolin-7-yl trifluoromethanesulfonate

595 mg of the starting phenol was suspended in 100 mL of anhydrous CH₂Cl₂ under argon. 224 μL of pyridine was added and the mixture stirred for 10 minutes at room temperature. The reaction mixture was cooled on ice and 846 μL of triflic anhydride was added in a dropwise manner while stirring. All the solids gradually dissolved and the mixture was stirred on ice for 1.5 hours at which point HPLC analysis indicated incomplete reaction. Added 225 μL pyridine and 450 μL triflic anhydride and stirred for 1 hour on ice. HPLC again indicated incomplete reaction. Added 750 μL of pyridine, 850 μL of triflic anhydride and 100 mL CH₂Cl₂. After an additional 1.5 hours on ice the reaction was determined to be complete by HPLC. Added 100 mL water and separated the layers. The organic phase washed with saturated aqueous sodium bicarbonate, dried over sodium sulfate, filtered and concentrated in vacuo. The residue was redissolved in a minimal volume of warm CH₂Cl₂ with a small amount of methanol and rapidly chromatographed over a pad of silica gel (6.5″ tall and 2″ wide). Elution was done with 100% CH₂Cl₂ at approximately 20 mL/min. Pure fractions, based on radioTLC analysis with 100% CH₂Cl₂, were pooled and concentrated to result in a white solid that was dried under vacuum. The yield was determined to be 87.4% (810.4 mg, 121 mCi) and the product characterized by R-HPLC and LCMS which showed >98% CP, >99% RCP and the desired parent ion.

### Preparation of 4-(3-chloro-4-(1-methyl-1H-imidazol-2-ylthio)phenylamino)-3-cyano-[¹⁴C]-6-methoxyquinolin-7-yl trifluoromethanesulfonate

To 810.4 mg of 4-chloro-3-cyano[¹⁴C]-6-methoxyquinolin-7-yl trifluoromethanesulfonate (121 mCi) was added 580 mg of 3-chloro-4-(1-methyl-1H-imidazol-2-ylthio)aniline, 381 mg of pyridine hydrochloride, and 45 mL of 2-ethoxyethanol. The reaction mixture was stirred in a oil bath at 110° C. for 5.5 hours with periodic HPLC checks. At this time it appeared that the reaction was not progressing further and impurity levels were starting to increase. The reaction mixture was slowly cooled to room temperature and stirred for 16 hours. The solvent was removed under high vacuum to give a brown oil. To this was added 5 mL of 2-ethoxyethanol. The mixture was stirred for 5 minutes at room temperature and for 30 minutes on ice. The resulting solids were filtered off and washed with ice cold 2-ethoxyethanol and the filtrate saved. The solids were suspended in 10 mL of saturated aqueous sodium bicarbonate and 100 mL ethyl acetate added. The resulting yellow slurry was stirred for 2 hours at room temperature, the solids filtered, washed with cold ethyl acetate and dried under vacuum (Crop 1). The filtrate was treated in the same manner using ½ volumes of bicarbonate and ethyl acetate, the layers were separated and the organic phase was dried over sodium sulfate, filtered and concentrated to dryness. The residue was dissolved/suspended in 5-6 ml of 2-ethoxyethanol, stirred for 30 minutes on ice and the resulting solids filtered and washed with cold ethoxylethanol. The resulting solids were dried overnight under vacuum and the filtrate concentrated to dryness and also dried overnight under vacuum (Crop 2). Crop 1 solids (816.7 mg, 78.5 mCi) were found to be >96% CP and RCP by HPLC. Crop 2 solids (16.5 mg) were found to be >88% CP and RCP. All solids were combined for the next step. The dried filtrate contained 296 mg of material that was approximately 80% pure and was set aside.

### Preparation and purification of 4-({3-chloro-4-[(1-methyl-1H-imidazol-2-yl)thio]phenyl}amino)-6-methoxy-7-(4-pyrrolidin-1-ylpiperidin-1-yl)quinoline-3-[¹⁴C]carbonitrile

To 4-(3-chloro-4-(1-methyl-1H-imidazol-2-ylthio)phenylamino)-3-cyano-[¹⁴C]-6-methoxyquinolin-7-yl trifluoromethanesulfonate was added 9 mL of anhydrous NMP and 9 mL anhydrous toluene under and argon atmosphere. 20 eq (2.52 g) of pyrrolidinyl-piperidine was added followed by 1.2 eq of anhydrous K₃PO₄ (208 mg), 0.05 eq of Pd₂(dba)₃ (37 mg) and 0.1 eq of di-t-butylphosphinobiphenyl (24 mg). The reaction mixture was heated in an oil bath at 85° C. and checked by HPLC after 4 hours. The reaction was not complete and additional K₃PO₄, catalyst and phosphine were added in amounts equal to that of the original addition. Heating was continued overnight (16 hours) and the mixture cooled to room temperature. HPLC analysis showed that little or no starting material remained and 4 products had been produced. The reaction mixture was filtered to remove the catalyst and the filter washed with methanol. The filtrate was concentrated to remove volatile solvents and then warmed to 75-80° C. Water (25% of total volume) was added in a dropwise manner and the mixture stirred for 30 minutes. To the warm mixture was added 2B-ethanol (30% by volume) and stirring continued for another 30 minutes. An equal volume of water was then added in a dropwise manner and the mixture stirred for 1 hour at 75-80° C. The mixture was slowly cooled to room temperature, then cooled in an ice bath and stirred for 40 minutes. The resulting slurry was filtered and washed with 2:1 water/2B-ethanol. The solids were dried overnight under vacuum and the filtrate stored at 4 C overnight.

The solids from each reaction were combined to give 49.6 mg of material. The filtrate that had been stored was cloudy and additional water was added while stirring until no more precipitate appeared. The precipitate was filtered off and treated as before. The resulting filtrate was extracted with ethyl acetate but upon combining the extracts, no desired material was seen by HPLC. The remaining aqueous NMP phase does contain some desired material but it is very dilute. All the precipitated solids were combined to give approximately 500 mg material that was 68% radiochemically pure but comprised approximately 33% by area. Purification was achieved by preparative HPLC using the following method:

YMC Pro C18 column (20×150 mm)

Solvent A=950/50/2.5 water/acetonitrile/trifluoroacetic acid

Solvent B=50/950/2.5 water/acetonitrile/trifluoroacetic acid

Flow rate=20 mL/min

Gradient:
hold 9% B for 3 min.
9% B→27% B over 50 min
27% B→95% B over 1 min
hold 95% B for 10 min

Diluent=aqueous acetic acid containing 10% NMP

Automatic fraction collection based on mass

Collected fractions were pooled and concentrated to remove the volatiles leaving an aqueous acidic solution, which was refrigerated until HPLC was complete. The aqueous product solution was made basic by slow addition of saturated aqueous sodium bicarbonate while stirring. The resulting slurry was refrigerated overnight, filtered and washed with water. The resulting pale yellow solid was dried under vacuum to yield 245 mg of product (23.4 mCi). This material was found to be slightly impure and was refluxed in 10 mL ethyl acetate for 30 min. After cooling to room temperature, the solid was filtered and then dried under vacuum to give 187 mg of material that still contained a 4.5% impurity. The solid was combined with 4 mL ethyl acetate and 4 ml of 2B-ethanol and warmed to reflux under argon. Another 5 mL of 2B-ethanol was added over 5 minutes at reflux, followed by 1 mL of water. The solution was refluxed for another 10 minutes and slowly cooled to room temperature. The resulting solution was stored at −20° C. overnight and a small amount of solid filtered off and washed with 6 mL of 2:1 water/2B-ethanol. The filtrate, which was now cloudy, was stirred at room temperature for 1 hour and a pale yellow solid filtered off. HPLC of this material showed it to be the desired product and contained <1% of the impurity. Additional water was added to the filtrate to generate a second crop in the same manner. Combined, the two crops totaled 105.7 mg. The specific activity was determined by MS to be 57.3 mCi/mmol. Chemical purity was determined to be >98% by HPLC peak area at 230 nm. Radiochemical purity was determined by R-HPLC and found to be >98%. Proton NMR analysis confirmed the structure of the product and also found 0.47% ethanol contamination. (J. Med. Chem. (2003) vol. 46, p 3197).

The above listed references are hereby incorporated by reference as they pertain to the synthesis of the disclosed compounds.

We claim:

1. A method for preparing radiolabeled 3-cyanoquinolines comprising the steps of reacting:
a. a compound of formula (IIa): 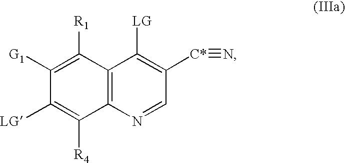
with a reagent of formula H-G₂ in the presence of a base to form the compound of formula (IIa): 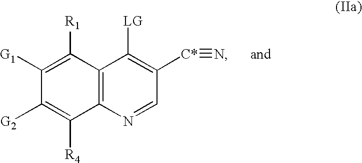
b. reacting the compound of formula (IIa) with a compound of formula H-Z-(CH₂)ₙ—X in the presence of a catalytic effective amount of an acid catalyst to produce a compound of formula (Ia): 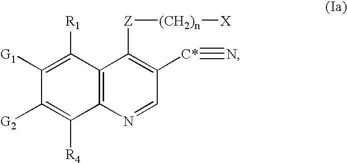
wherein:
LG′ is —O-triflate or —O-mesylate;
X is a cycloalkyl of 3 to 7 carbon atoms, which may be optionally substituted with one or more alkyl of 1 to 6 carbon atoms, a pyridinyl, a pyrimidinyl, or a phenyl ring; wherein the pyridinyl, pyrimidinyl, or phenyl ring may be optionally mono-, di-, or tri-substituted with substituents selected from a group consisting of a halogen, an alkyl of 1-6 carbon atoms, an alkenyl of 2-6 carbon atoms, an alkynyl of 2-6 carbon atoms, azido, a hydroxyalkyl of 1-6 carbon atoms, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a benzoyl, an amino, an alkylamino of 1-6 carbon atoms, a dialkylamino of 2 to 12 carbon atoms, an alkanoylamino of 1-6 carbon atoms, an alkenoylamino of 3-8 carbon atoms, an alkynoylamino of 3-8 carbon atoms, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, a N,N-dialkylaminoalkyl of 3-10 carbon atoms, a N-alkylaminoalkoxy of 3-9 carbon atoms, a N,N-dialkylaminoalkoxy of 4-10 carbon atoms, mercapto, methylmercapto and benzoylamino;
a bicyclic aryl or bicyclic heteroaryl ring system of 8 to 12 atoms where the bicyclic heteroaryl ring contains 1 to 4 heteroatoms selected from N, O, and S, wherein the bicyclic aryl or bicyclic heteroaryl ring may be optionally mono- di-, tri, or tetra-substituted with a substituent comprising a halogen, an oxo, a thiocarbonyl, an alkyl of 1-6 carbon atoms, an alkenyl of 2-6 carbon atoms, an alkynyl of 2-6 carbon atoms, azido, a hydroxyalkyl of 1-6 carbon atoms, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, a N,N-dialkylaminoalkyl of 3-10 carbon atoms, an N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, and a benzoylamino;
a radical of the form: 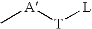
wherein;
A′ is a pyridinyl, a pyrimidinyl, or a phenyl ring; wherein the pyridinyl, pyrimidinyl, or phenyl ring may be optionally mono- or di-substituted with a substituent comprising an alkyl of 1-6 carbon atoms, an alkenyl of 2-6 carbon atoms, an alkynyl of 2-6 carbon atoms, azido, a hydroxyalkyl of 1-6 carbon atoms, a halogen, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, a N-alkylaminoalkyl of 2-9 carbon atoms, a N,N-dialkylaminoalkyl of 3-10 carbon atoms, an N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, a alkenoyloxy of 3-8 carbon atoms, an alkynoyloxy of 3-8 carbon atoms, a carbamoyl, a N-alkylcarbamoyl of 2-7 carbon atoms, a N,N-dialkylcarbamoyl of 3-13 carbon atoms, and benzoylamino;
T is substituted at a carbon of the pyridinyl, pyrimidinyl, or phenyl ring with —NH(CH₂)ₘ—, —O(CH₂)ₘ—, —S(CH₂)ₘ—, —NR(CH₂)ₘ—, —(CH₂)ₘ—, —(CH₂)ₘNH—, —(CH₂)ₘO—, —(CH₂)ₘS—, —SO(CH₂)ₘ—, —SO₂(CH₂)ₘ—, —CO(CH₂)ₘ—, —(CH₂)ₘCO—, —(CH₂)ₘSO—, —(CH₂)ₘSO₂— or —(CH₂)ₘNR—;
L is an imidazole or a phenyl ring wherein the imidazole or phenyl ring are optionally substituted at a carbon or nitrogen with one, two, or three substituents comprising an alkyl of 1-6 carbon atoms, an alkenyl of 2-6 carbon atoms, an alkynyl of 2-6 carbon atoms, azido, a hydroxyalkyl of 1-6 carbon atoms, a halogen, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, an N,N-dialkylaminoalkyl of 3-10 carbon atoms, a N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, a benzoylamino, a 5- or 6-membered heteroaryl ring where the heteroaryl ring contains 1 to 3 heteroatoms selected from N, O, and S and where the heteroaryl ring may be optionally mono- or di-substituted with a substituent comprising a halogen, an oxo, a thiocarbonyl, an alkyl of 1-6 carbon atoms, an alkenyl of 2-6 carbon atoms, an alkynyl of 2-6 carbon atoms, azido, a hydroxyalkyl of 1-6 carbon atoms, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, a N,N-dialkylaminoalkyl of 3-10 carbon atoms, a N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, and a benzoylamino;
LG is selected from halo, O-triflate, and O-mesylate;
Z is —NH—, —O—, —S—, or —NR—;
R is alkyl of 1-6 carbon atoms;
G₂ is —O-alkyl, —O-phenyl, —O-benzyl, alkylamino of 1-6 carbon atoms, dialkylamino of 2 to 12 carbon atoms, phenylamino, benzylamino, heterocycloalkyl, heterocycloalkyl-heterocycloalkyl, where the heterocycloalkyl group or the heterocycloalkyl-heterocycloalkyl group contain a secondary amino functionality in the ring;
G₁, R₁ and R₄ are each, independently, a hydrogen, a halogen, a hydroxy, an amino, a hydroxyamino, a trifluoromethyl, a trifluoromethoxy, a mercapto, an alkyl of 1-6 carbon atoms, a cycloalkyl of 3-8 carbon atoms, an alkenyl of 2-6 carbon atoms, an alkynyl of 2-6 carbon atoms, an alkenyloxy of 2-6 carbon atoms, an alkynyloxy of 2-6 carbon atoms, a hydroxyalkyl of 1-6 carbon atoms, a mercaptoalkyl of 1-6 carbon atoms, a halomethyl, an alkoxymethyl of 2-7 carbon atoms, an alkoxy of 1-6 carbon atoms, a 2-methoxyethoxy, a 2-(2-methoxyethoxy)ethoxy, a cycloalkoxy of 3-8 carbon atoms, an alkylthio of 1-6 carbon atoms, a cycloalkylthio of 3-8 carbon atoms, an alkylsulfinyl of 1-6 carbon atoms, an alkylsulfonyl of 1-6 carbon atoms, an alkylsulfonamido of 1-6 carbon atoms, an alkenylsulfonamido of 2-6 carbon atoms, an alkynylsulfonamido of 2-6 carbon atoms, an alkylcarboxamido of 2-7 carbon atoms, a (N-alkyl)alkylcarboxamido of 3-13 carbon atoms, an alkenylcarboxamido of 3-7 carbon atoms, an (N-alkyl)alkenylcarboxamido of 4-13 carbon atoms, an alkynylcarboxamido of 3-7 carbon atoms, an (N-alkyl)alkynylcarboxamido of 4-13 carbon atoms, cyano, nitro, carboxy, a alkoxycarbonyl of 2-7 carbon atoms, an alkanoyl of 2-7 carbon atoms, an alkenoyl of 3-7 carbon atoms, a N-alkyl-N-alkenylamino of 4 to 12 carbon atoms, an N,N-dialkenylamino of 6-12 carbon atoms, phenylamino, benzylamino, phenoxy, phenyl, thiophenoxy, benzyl, an alkylamino of 1-6 carbon atoms, an alkanoyloxy of 2-7 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, a dialkylamino of 2 to 12 carbon atoms, an alkanoyloxymethyl group of 2-7 carbon atoms, an alkenoyloxymethyl group of 2-7 carbon atoms, an alkynoyloxymethyl group of 2-7 carbon atoms, azido, benzoyl, a carboxyalkyl of 2-7 carbons, and a carboalkoxyalkyl of 3-8 carbon atoms,
—NR₆C(O)H; —N(C(O)R₆)C(O)R₆;
—Y—(C(R₆)₂)ₚ-Het1;
—Y—(C(R₆)₂)ₚ-Het1-(C(R₆)₂)_{q}-Z¹-(C(R₆)₂)ᵣ-Het2;
Het1 is a 3-8 membered saturated heterocyclic ring containing one or more nitrogen, oxygen or sulfur atoms such as, but not limited to morpholine, thiomorpholine, thiomorpholine S-oxide, thiomorpholine S,S-dioxide, piperidine, pyrrolidine, aziridine, piperazine, tetrahydrothiophene, tetrahydrofuran, dioxane, 1,3-dioxolane pyrrole, tetrahydropyran, and diazepan; wherein Het1 is optionally mono- or di-substituted on a carbon or a nitrogen with R₆; optionally mono- or di-substituted on a carbon with hydroxy, —N(R₆)₂, or —OR₆; optionally mono or di-substituted on a carbon with the mono-valent radicals —(C(R₆)₂)ₛOR₆ or —[(C(R₆)₂)ₛN(R₆)₂]; or optionally mono or di-substituted on a saturated carbon with divalent radicals ═O or —O(C(R₆)₂)ₛO—;
Het2 is a heteroaryl selected from the group comprising morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane pyrrole, and tetrahydropyran; wherein Het2 is optionally mono- or di-substituted on a carbon or a nitrogen with R₆; optionally mono- or di-substituted on a carbon with hydroxy, —N(R₆)₂, or —OR₆; optionally mono or di-substituted on a carbon with the mono-valent radicals —(C(R₆)₂)ₛOR₆ or —[(C(R₆)₂)ₛN(R₆)₂]; or optionally mono or di-substituted on a saturated carbon with divalent radicals ═O or —O(C(R₆)₂)ₛO—;
R₆ is hydrogen, an alkyl of 1-6 carbon atoms, an alkenyl of 2-6 carbon atoms, an alkynyl of 2-6 carbon atoms, a cycloalkyl of 3-6 carbon atoms, an alkanoyl of 2-7 carbon atoms, a carbamoylalkyl of 2-7 carbon atoms, a hydroxyalkyl of 1-6 carbon atoms, a hydroxycycloalkyl of 3-6 carbon atoms, a carboxyalkyl of 2-7 carbon atoms, pyrrolidine, piperidine, or imidazole optionally substituted with methyl;
a phenyl optionally mono-, di-, or tri-substituted with halogen, an alkoxy of 1-6 carbon atoms, trifluoromethyl, amino, an alkylamino of 1-3 carbon atoms, a dialkylamino of 2-6 carbon atoms, nitro, cyano, azido, halomethyl, an alkoxymethyl of 2-7 carbon atoms, an alkanoyloxymethyl of 2-7 carbon atoms, an alkylthio of 1-6 carbon atoms, hydroxy, a carboxyl, a alkoxycarbonyl of 2-7 carbon atoms, phenoxy, phenyl, thiophenoxy, benzoyl, benzyl, phenylamino, benzylamino, an alkanoylamino of 1-6 carbon atoms or alkyl of 1-6 carbon atoms;
Y is O, S, —NR₆C(O)—, —C(O)NR₆—, NR₆ or a bond;
Z¹ is O, S, —NR₆C(O)—, —C(O)NR₆—, NR₆ or a bond;
p is 0-4;
q is 0-4;
r is 0-4;
s is 1-6; and
at least one of the groups, R₁, R₂, R₃ or R₄ is consisting of —Y—(C(R₆)₂)ₚ-Het1-(C(R₆)₂)_{q}-Z-(C(R₆)₂)ᵣ-Het2;
m is 0-3; and
n is 0-1.

2. The method of claim 1, wherein the acid is CF₃SO₃H.

3. The method of claim 1, wherein the base is K₃PO₄.

4. The method of claim 1, wherein LG is Cl.

5. The method of claim 1, wherein LG′ is O-triflate.

6. The method of claim 1, wherein the compound formed is 4-({3-chloro-4-[(1-methyl-1H-imidazol-2-yl)thio]phenyl}amino)-6-methoxy-7-(4-pyrrolidin-1-ylpiperidin-1-yl)quinoline-3-[¹⁴C]carbonitrile.

7. A method for preparing radiolabeled 3-cyanoquinolines comprising the step of reacting
(i) a compound of formula H-G₂′, and
(ii) a 3-cyanoquinoline intermediate having formula (IIb): 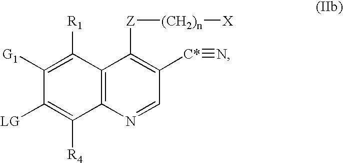
in the presence of a base to produce a compound of formula (Ib): 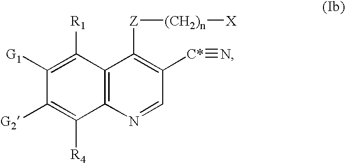
wherein:
G₂′ is Y′—R^{Y}, 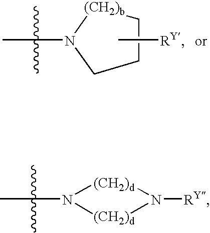
where b is 0, 1, or 2, and d is 1 or 2;
Y′ is selected from —NR′—, —O— and —S—, where R′ is alkyl of 1 to 6 carbon atoms;
R^{Y} is selected from alkyl, alkenyl, alkynyl, aryl, benzyl, heterocycloalkyl, -aryl-aryl, -aryl-heterocycloalkyl, -heterocycloalkyl-aryl, and heterocycloalkyl-heterocycloalkyl;
R^{Y′} is selected from alkyl, halo, haloalkyl, alkenyl, alkynyl, aryl, benzyl, heterocycloalkyl, alkoxy, haloalkoxy, aryloxy, —O—CH₂-phenyl, —O-heterocycloalkyl, dialkylamino, —N(alkyl)(aryl), diarylamino, —S-alkyl, —S-aryl, —S-heterocycloalkyl, and CN;
R^{Y″} is selected from alkyl, halo, haloalkyl, alkenyl, alkynyl, aryl, benzyl, and heterocycloalkyl, any of which may be substituted with suitable substituents;
X is a cycloalkyl of 3 to 7 carbon atoms, which may be optionally substituted with one or more alkyl of 1 to 6 carbon atoms, a pyridinyl, a pyrimidinyl, or a phenyl ring; wherein the pyridinyl, pyrimidinyl, or phenyl ring may be optionally mono-, di-, or tri-substituted with substituents selected from a group consisting of a halogen, an alkyl of 1-6 carbon atoms, an alkenyl of 2-6 carbon atoms, an alkynyl of 2-6 carbon atoms, azido, a hydroxyalkyl of 1-6 carbon atoms, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a benzoyl, an amino, an alkylamino of 1-6 carbon atoms, a dialkylamino of 2 to 12 carbon atoms, an alkanoylamino of 1-6 carbon atoms, an alkenoylamino of 3-8 carbon atoms, an alkynoylamino of 3-8 carbon atoms, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, a N,N-dialkylaminoalkyl of 3-10 carbon atoms, a N-alkylaminoalkoxy of 3-9 carbon atoms, a N,N-dialkylaminoalkoxy of 4-10 carbon atoms, mercapto, methylmercapto and benzoylamino;
a bicyclic aryl or bicyclic heteroaryl ring system of 8 to 12 atoms where the bicyclic heteroaryl ring contains 1 to 4 heteroatoms selected from N, O, and S, wherein the bicyclic aryl or bicyclic heteroaryl ring may be optionally mono- di-, tri, or tetra-substituted with a substituent comprising a halogen, an oxo, a thiocarbonyl, an alkyl of 1-6 carbon atoms, an alkenyl of 2-6 carbon atoms, an alkynyl of 2-6 carbon atoms, azido, a hydroxyalkyl of 1-6 carbon atoms, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, a N,N-dialkylaminoalkyl of 3-10 carbon atoms, an N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, and a benzoylamino;
a radical of the form: 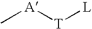
wherein;
A′ is a pyridinyl, a pyrimidinyl, or a phenyl ring; wherein the pyridinyl, pyrimidinyl, or phenyl ring may be optionally mono- or di-substituted with a substituent comprising an alkyl of 1-6 carbon atoms, an alkenyl of 2-6 carbon atoms, an alkynyl of 2-6 carbon atoms, azido, a hydroxyalkyl of 1-6 carbon atoms, a halogen, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, a N-alkylaminoalkyl of 2-9 carbon atoms, a N,N-dialkylaminoalkyl of 3-10 carbon atoms, an N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, a alkenoyloxy of 3-8 carbon atoms, an alkynoyloxy of 3-8 carbon atoms, a carbamoyl, a N-alkylcarbamoyl of 2-7 carbon atoms, a N,N-dialkylcarbamoyl of 3-13 carbon atoms, and benzoylamino;
T is substituted at a carbon of the pyridinyl, pyrimidinyl, or phenyl ring with —NH(CH₂)ₘ—, —O(CH₂)ₘ—, —S(CH₂)ₘ—, —NR(CH₂)ₘ—, —(CH₂)ₘ—, —(CH₂)ₘNH—, —(CH₂)ₘO—, —(CH₂)ₘS—, —SO(CH₂)ₘ—, —SO₂(CH₂)ₘ—, —CO(CH₂)ₘ—, —(CH₂)ₘCO—, —(CH₂)ₘSO—, —(CH₂)ₘSO₂— or —(CH₂)ₘNR—;
L is an imidazole or a phenyl ring wherein the imidazole or phenyl ring are optionally substituted at a carbon or nitrogen with one, two, or three substituents comprising an alkyl of 1-6 carbon atoms, an alkenyl of 2-6 carbon atoms, an alkynyl of 2-6 carbon atoms, azido, a hydroxyalkyl of 1-6 carbon atoms, a halogen, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, an N,N-dialkylaminoalkyl of 3-10 carbon atoms, a N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, a benzoylamino, a 5- or 6-membered heteroaryl ring where the heteroaryl ring contains 1 to 3 heteroatoms selected from N, O, and S and where the heteroaryl ring may be optionally mono- or di-substituted with a substituent comprising a halogen, an oxo, a thiocarbonyl, an alkyl of 1-6 carbon atoms, an alkenyl of 2-6 carbon atoms, an alkynyl of 2-6 carbon atoms, azido, a hydroxyalkyl of 1-6 carbon atoms, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, a N,N-dialkylaminoalkyl of 3-10 carbon atoms, a N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, and a benzoylamino;
LG is selected from halo, —O-triflate and —O-mesylate;
Z is —NH—, —O—, —S—, or —NR—,
R is alkyl of 1-6 carbon atoms,
G₁, R₁, and R₄ are each, independently, a hydrogen, a halogen, a hydroxy, an amino, a hydroxyamino, a trifluoromethyl, a trifluoromethoxy, a mercapto, an alkyl of 1-6 carbon atoms, a cycloalkyl of 3-8 carbon atoms, an alkenyl of 2-6 carbon atoms, an alkynyl of 2-6 carbon atoms, an alkenyloxy of 2-6 carbon atoms, an alkynyloxy of 2-6 carbon atoms, a hydroxyalkyl of 1-6 carbon atoms, a mercaptoalkyl of 1-6 carbon atoms, a halomethyl, an alkoxymethyl of 2-7 carbon atoms, an alkoxy of 1-6 carbon atoms, a 2-methoxyethoxy, a 2-(2-methoxyethoxy)ethoxy, a cycloalkoxy of 3-8 carbon atoms, an alkylthio of 1-6 carbon atoms, a cycloalkylthio of 3-8 carbon atoms, an alkylsulfinyl of 1-6 carbon atoms, an alkylsulfonyl of 1-6 carbon atoms, an alkylsulfonamido of 1-6 carbon atoms, an alkenylsulfonamido of 2-6 carbon atoms, an alkynylsulfonamido of 2-6 carbon atoms, an alkylcarboxamido of 2-7 carbon atoms, a (N-alkyl)alkylcarboxamido of 3-13 carbon atoms, an alkenylcarboxamido of 3-7 carbon atoms, an (N-alkyl)alkenylcarboxamido of 4-13 carbon atoms, an alkynylcarboxamido of 3-7 carbon atoms, an (N-alkyl)alkynylcarboxamido of 4-13 carbon atoms, cyano, nitro, carboxy, a alkoxycarbonyl of 2-7 carbon atoms, an alkanoyl of 2-7 carbon atoms, an alkenoyl of 3-7 carbon atoms, a N-alkyl-N-alkenylamino of 4 to 12 carbon atoms, an N,N-dialkenylamino of 6-12 carbon atoms, phenylamino, benzylamino, phenoxy, phenyl, thiophenoxy, benzyl, an alkylamino of 1-6 carbon atoms, an alkanoyloxy of 2-7 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, a dialkylamino of 2 to 12 carbon atoms, an alkanoyloxymethyl group of 2-7 carbon atoms, an alkenoyloxymethyl group of 2-7 carbon atoms, an alkynoyloxymethyl group of 2-7 carbon atoms, azido, benzoyl, a carboxyalkyl of 2-7 carbons, and a carboalkoxyalkyl of 3-8 carbon atoms,
—NR₆C(O)H; —N(C(O)R₆)C(O)R₆;
—Y—(C(R₆)₂)ₚ-Het1;
—Y—(C(R₆)₂)ₚ-Het1-(C(R₆)₂)_{q}-Z¹-(C(R₆)₂)ᵣ-Het2;
Het1 is a 3-8 membered saturated heterocyclic ring containing one or more nitrogen, oxygen or sulfur atoms such as, but not limited to morpholine, thiomorpholine, thiomorpholine S-oxide, thiomorpholine S,S-dioxide, piperidine, pyrrolidine, aziridine, piperazine, tetrahydrothiophene, tetrahydrofuran, dioxane, 1,3-dioxolane pyrrole, tetrahydropyran, and diazepan; wherein Het1 is optionally mono- or di-substituted on a carbon or a nitrogen with R₆; optionally mono- or di-substituted on a carbon with hydroxy, —N(R₆)₂, or —OR₆; optionally mono or di-substituted on a carbon with the mono-valent radicals —(C(R₆)₂)ₛOR₆ or —[(C(R₆)₂)ₛN(R₆)₂]; or optionally mono or di-substituted on a saturated carbon with divalent radicals ═O or —O(C(R₆)₂)ₛO—;
Het2 is a heteroaryl selected from the group comprising morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane pyrrole, and tetrahydropyran; wherein Het2 is optionally mono- or di-substituted on a carbon or a nitrogen with R₆; optionally mono- or di-substituted on a carbon with hydroxy, —N(R₆)₂, or —OR₆; optionally mono or di-substituted on a carbon with the mono-valent radicals —(C(R₆)₂)ₛOR₆ or —[(C(R₆)₂)ₛN(R₆)₂]; or optionally mono or di-substituted on a saturated carbon with divalent radicals ═O or —O(C(R₆)₂)ₛO—;
R₆ is hydrogen, an alkyl of 1-6 carbon atoms, an alkenyl of 2-6 carbon atoms, an alkynyl of 2-6 carbon atoms, a cycloalkyl of 3-6 carbon atoms, an alkanoyl of 2-7 carbon atoms, a carbamoylalkyl of 2-7 carbon atoms, a hydroxyalkyl of 1-6 carbon atoms, a hydroxycycloalkyl of 3-6 carbon atoms, a carboxyalkyl of 2-7 carbon atoms, pyrrolidine, piperidine, or imidazole optionally substituted with methyl;
a phenyl optionally mono-, di-, or tri-substituted with halogen, an alkoxy of 1-6 carbon atoms, trifluoromethyl, amino, an alkylamino of 1-3 carbon atoms, a dialkylamino of 2-6 carbon atoms, nitro, cyano, azido, halomethyl, an alkoxymethyl of 2-7 carbon atoms, an alkanoyloxymethyl of 2-7 carbon atoms, an alkylthio of 1-6 carbon atoms, hydroxy, a carboxyl, a alkoxycarbonyl of 2-7 carbon atoms, phenoxy, phenyl, thiophenoxy, benzoyl, benzyl, phenylamino, benzylamino, an alkanoylamino of 1-6 carbon atoms or alkyl of 1-6 carbon atoms;
Y is O, S, —NR₆C(O)—, —C(O)NR₆—, NR₆ or a bond;
Z¹ is O, S, —NR₆C(O)—, —C(O)NR₆—, NR₆ or a bond;
p is 0-4;
q is 0-4;
r is 0-4;
s is 1-6;
m is 0-3; and
n is 0-1.

8. The method of claim 7, further comprising the step of reacting a compound of formula (IIIb): 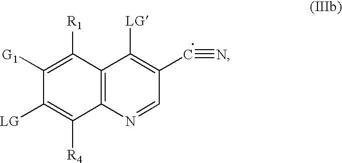
wherein LG′ is halo with a reagent of formula H-Z-(CH₂)ₙ—X in an acidic environment to form a compound of formula (IIb).

9. The method of claim 8, wherein X is A′-T-L, and L is 1-methyl-1H-imidazolyl.

10. The method of claim 7, wherein R₁ is H.

11. The method of claim 7, wherein R₄ is H.

12. The method of claim 8, wherein pyridine HCl is used to create the acidic environment.

13. The method of claim 7, wherein the base is K₃PO₄.

14. The method of claim 7, wherein the compound formed is 4-({3-chloro-4-[(1-methyl-1H-imidazol-2-yl)thio]phenyl}amino)-6-methoxy-7-(4-pyrrolidin-1-ylpiperidin-1-yl)quinoline-3-[¹⁴C]carbonitrile.

15. The method of claim 8, further comprising the step of reacting a compound of formula (IV): 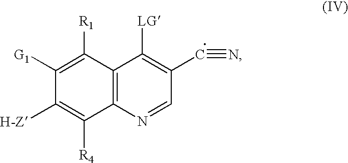
with a reagent effective to form a compound of formula (IIIb): 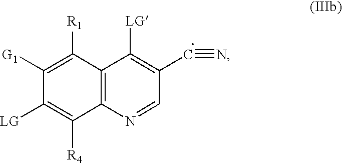
wherein Z′ is O or S.

16. The method of claim 15, further comprising the step of deprotecting a compound of formula (V): 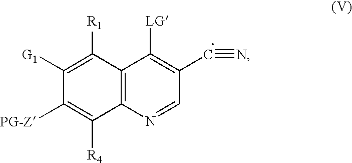
to form a compound of formula (IV): 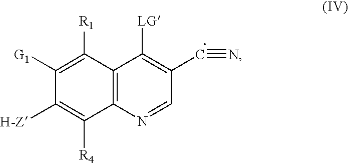
wherein PG is selected from benzyl, acetyl, methoxymethyl, benzyloxymethyl, methoxyethoxymethyl, 4-methoxybenzyl, and —C(O)O—CH₂—CCl₃.

17. The method of claim 16, further comprising the steps of:
a. cyclizing a compound of formula (VIIb): 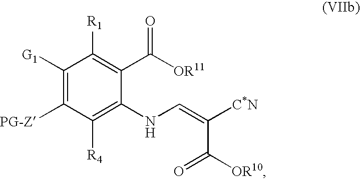
to form a compound of formula (VI): 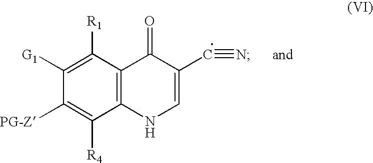
b. reacting the compound of formula (VI) with a reagent effective to form a compound of formula (V): 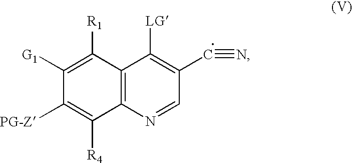
wherein R¹⁰ is selected from alkyl, aryl, cycloalkyl and heterocycloalkyl and R¹¹ is selected from alkyl, aryl, cycloalkyl and heterocycloalkyl.

18. The method of claim 17, wherein Z′ is O.

19. A method for preparing radiolabeled compounds of formula (VII): 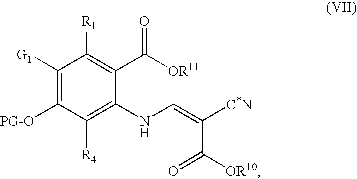
comprising the step of reacting:
(i) a compound of formula R¹⁰O—C(O)—CH₂—C*N, and
(ii) an intermediate having formula (VIII): 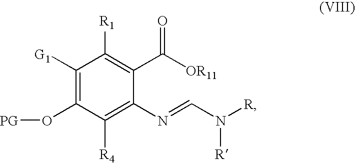
wherein:
PG is selected from benzyl, acetyl, methoxymethyl, benzyloxymethyl, methoxyethoxymethyl, 4-methoxybenzyl, and —C(O)O—CH₂—CCl₃;
R and R′ are independently alkyl, aryl or benzyl;
R¹⁰ is selected from alkyl, aryl, cycloalkyl and heterocycloalkyl;
R¹¹ is selected from alkyl, aryl, cycloalkyl and heterocycloalkyl;
G₁, R₁, and R₄ are each, independently, a hydrogen, a halogen, a hydroxy, an amino, a hydroxyamino, a trifluoromethyl, a trifluoromethoxy, a mercapto, an alkyl of 1-6 carbon atoms, a cycloalkyl of 3-8 carbon atoms, an alkenyl of 2-6 carbon atoms, an alkynyl of 2-6 carbon atoms, an alkenyloxy of 2-6 carbon atoms, an alkynyloxy of 2-6 carbon atoms, a hydroxyalkyl of 1-6 carbon atoms, a mercaptoalkyl of 1-6 carbon atoms, a halomethyl, an alkoxymethyl of 2-7 carbon atoms, an alkoxy of 1-6 carbon atoms, a 2-methoxyethoxy, a 2-(2-methoxyethoxy)ethoxy, a cycloalkoxy of 3-8 carbon atoms, an alkylthio of 1-6 carbon atoms, a cycloalkylthio of 3-8 carbon atoms, an alkylsulfinyl of 1-6 carbon atoms, an alkylsulfonyl of 1-6 carbon atoms, an alkylsulfonamido of 1-6 carbon atoms, an alkenylsulfonamido of 2-6 carbon atoms, an alkynylsulfonamido of 2-6 carbon atoms, an alkylcarboxamido of 2-7 carbon atoms, a (N-alkyl)alkylcarboxamido of 3-13 carbon atoms, an alkenylcarboxamido of 3-7 carbon atoms, an (N-alkyl)alkenylcarboxamido of 4-13 carbon atoms, an alkynylcarboxamido of 3-7 carbon atoms, an (N-alkyl)alkynylcarboxamido of 4-13 carbon atoms, cyano, nitro, carboxy, a alkoxycarbonyl of 2-7 carbon atoms, an alkanoyl of 2-7 carbon atoms, an alkenoyl of 3-7 carbon atoms, a N-alkyl-N-alkenylamino of 4 to 12 carbon atoms, an N,N-dialkenylamino of 6-12 carbon atoms, phenylamino, benzylamino, phenoxy, phenyl, thiophenoxy, benzyl, an alkylamino of 1-6 carbon atoms, an alkanoyloxy of 2-7 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, a dialkylamino of 2 to 12 carbon atoms, an alkanoyloxymethyl group of 2-7 carbon atoms, an alkenoyloxymethyl group of 2-7 carbon atoms, an alkynoyloxymethyl group of 2-7 carbon atoms, azido, benzoyl, a carboxyalkyl of 2-7 carbons, and a carboalkoxyalkyl of 3-8 carbon atoms,
—NR₆C(O)H; —N(C(O)R₆)C(O)R₆;
—Y—(C(R₆)₂)ₚ-Het1;
—Y—(C(R₆)₂)ₚ-Het1-(C(R₆)₂)_{q}-Z¹-(C(R₆)₂)ᵣ-Het2;
Het1 is a 3-8 membered saturated heterocyclic ring containing one or more nitrogen, oxygen or sulfur atoms such as, but not limited to morpholine, thiomorpholine, thiomorpholine S-oxide, thiomorpholine S,S-dioxide, piperidine, pyrrolidine, aziridine, piperazine, tetrahydrothiophene, tetrahydrofuran, dioxane, 1,3-dioxolane pyrrole, tetrahydropyran, and diazepan; wherein Het1 is optionally mono- or di-substituted on a carbon or a nitrogen with R₆; optionally mono- or di-substituted on a carbon with hydroxy, —N(R₆)₂, or —OR₆; optionally mono or di-substituted on a carbon with the mono-valent radicals —(C(R₆)₂)ₛOR₆ or —[(C(R₆)₂)ₛN(R₆)₂]; or optionally mono or di-substituted on a saturated carbon with divalent radicals ═O or —O(C(R₆)₂)ₛO—;
Het2 is a heteroaryl selected from the group comprising morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane pyrrole, and tetrahydropyran; wherein Het2 is optionally mono- or di-substituted on a carbon or a nitrogen with R₆; optionally mono- or di-substituted on a carbon with hydroxy, —N(R₆)₂, or —OR₆; optionally mono or di-substituted on a carbon with the mono-valent radicals —(C(R₆)₂)ₛOR₆ or —[(C(R₆)₂)ₛN(R₆)₂]; or optionally mono or di-substituted on a saturated carbon with divalent radicals ═O or —O(C(R₆)₂)ₛO—;
R₆ is hydrogen, an alkyl of 1-6 carbon atoms, an alkenyl of 2-6 carbon atoms, an alkynyl of 2-6 carbon atoms, a cycloalkyl of 3-6 carbon atoms, an alkanoyl of 2-7 carbon atoms, a carbamoylalkyl of 2-7 carbon atoms, a hydroxyalkyl of 1-6 carbon atoms, a hydroxycycloalkyl of 3-6 carbon atoms, a carboxyalkyl of 2-7 carbon atoms, pyrrolidine, piperidine, or imidazole optionally substituted with methyl;
a phenyl optionally mono-, di-, or tri-substituted with halogen, an alkoxy of 1-6 carbon atoms, trifluoromethyl, amino, an alkylamino of 1-3 carbon atoms, a dialkylamino of 2-6 carbon atoms, nitro, cyano, azido, halomethyl, an alkoxymethyl of 2-7 carbon atoms, an alkanoyloxymethyl of 2-7 carbon atoms, an alkylthio of 1-6 carbon atoms, hydroxy, a carboxyl, a alkoxycarbonyl of 2-7 carbon atoms, phenoxy, phenyl, thiophenoxy, benzoyl, benzyl, phenylamino, benzylamino, an alkanoylamino of 1-6 carbon atoms or alkyl of 1-6 carbon atoms;
Y is O, S, —NR₆C(O)—, —C(O)NR₆—, NR₆ or a bond;
Z¹ is O, S, —NR₆C(O)—, —C(O)NR₆—, NR₆ or a bond;
p is 0-4;
q is 0-4;
r is 0-4; and
s is 1-6.

20. The method of claim 19, wherein R and R′ are alkyl.

21. The method of claim 20, wherein R and R′ are methyl.

22. The method of claim 19, wherein G₁, R₁, and R₄ are each, independently, hydrogen, halogen, alkyl of 1-6 carbon atoms, hydroxymethyl, halomethyl, alkoxy of 1-6 carbon atoms, alkylthio of 1-6 carbon atoms, trifluoromethyl, trifluoromethoxy, cyano, nitro, phenoxy, phenyl, thiophenoxy, benzyl, alkylamino of 1-6 carbon atoms, or dialkylamino of 2 to 12 carbon atoms.

23. The method of claim 22, wherein G₁, R₁, and R₄ are each, independently, hydrogen, alkyl of 1-6 carbon atoms, trifluoromethyl, trifluoromethoxy or alkoxy of 1-6 carbon atoms.

24. The method of claim 23, wherein R₁ is hydrogen.

25. The method of claim 23, wherein R₄ is hydrogen.

26. The method of claim 23, wherein G₁ is alkoxy of 1-6 carbon atoms.

27. The method of claim 26, wherein G₁ is methoxy.

28. The method of claim 19, wherein PG is benzyl.

29. The method of claim 19, wherein the reaction occurs at about 25° C. or less.

30. A radiolabeled compound of formula (VII): 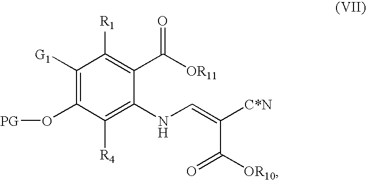
wherein:
PG is selected from benzyl, acetyl, methoxymethyl, benzyloxymethyl, methoxyethoxymethyl, 4-methoxybenzyl, and —C(O)O—CH₂—CCl₃;
R and R′ are independently alkyl, aryl or benzyl;
R¹⁰ is selected from alkyl, aryl, cycloalkyl and heterocycloalkyl;
R¹¹ is selected from alkyl, aryl, cycloalkyl and heterocycloalkyl;
G₁, R₁, and R₄ are each, independently, a hydrogen, a halogen, a hydroxy, an amino, a hydroxyamino, a trifluoromethyl, a trifluoromethoxy, a mercapto, an alkyl of 1-6 carbon atoms, a cycloalkyl of 3-8 carbon atoms, an alkenyl of 2-6 carbon atoms, an alkynyl of 2-6 carbon atoms, an alkenyloxy of 2-6 carbon atoms, an alkynyloxy of 2-6 carbon atoms, a hydroxyalkyl of 1-6 carbon atoms, a mercaptoalkyl of 1-6 carbon atoms, a halomethyl, an alkoxymethyl of 2-7 carbon atoms, an alkoxy of 1-6 carbon atoms, a 2-methoxyethoxy, a 2-(2-methoxyethoxy)ethoxy, a cycloalkoxy of 3-8 carbon atoms, an alkylthio of 1-6 carbon atoms, a cycloalkylthio of 3-8 carbon atoms, an alkylsulfinyl of 1-6 carbon atoms, an alkylsulfonyl of 1-6 carbon atoms, an alkylsulfonamido of 1-6 carbon atoms, an alkenylsulfonamido of 2-6 carbon atoms, an alkynylsulfonamido of 2-6 carbon atoms, an alkylcarboxamido of 2-7 carbon atoms, a (N-alkyl)alkylcarboxamido of 3-13 carbon atoms, an alkenylcarboxamido of 3-7 carbon atoms, an (N-alkyl)alkenylcarboxamido of 4-13 carbon atoms, an alkynylcarboxamido of 3-7 carbon atoms, an (N-alkyl)alkynylcarboxamido of 4-13 carbon atoms, cyano, nitro, carboxy, a alkoxycarbonyl of 2-7 carbon atoms, an alkanoyl of 2-7 carbon atoms, an alkenoyl of 3-7 carbon atoms, a N-alkyl-N-alkenylamino of 4 to 12 carbon atoms, an N,N-dialkenylamino of 6-12 carbon atoms, phenylamino, benzylamino, phenoxy, phenyl, thiophenoxy, benzyl, an alkylamino of 1-6 carbon atoms, an alkanoyloxy of 2-7 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, a dialkylamino of 2 to 12 carbon atoms, an alkanoyloxymethyl group of 2-7 carbon atoms, an alkenoyloxymethyl group of 2-7 carbon atoms, an alkynoyloxymethyl group of 2-7 carbon atoms, azido, benzoyl, a carboxyalkyl of 2-7 carbons, and a carboalkoxyalkyl of 3-8 carbon atoms,
—NR₆C(O)H; —N(C(O)R₆)C(O)R₆;
—Y—(C(R₆)₂)ₚ-Het1;
—Y—(C(R₆)₂)ₚ-Het1-(C(R₆)₂)_{q}-Z¹-(C(R₆)₂)ᵣ-Het2;
Het1 is a 3-8 membered saturated heterocyclic ring containing one or more nitrogen, oxygen or sulfur atoms such as, but not limited to morpholine, thiomorpholine, thiomorpholine S-oxide, thiomorpholine S,S-dioxide, piperidine, pyrrolidine, aziridine, piperazine, tetrahydrothiophene, tetrahydrofuran, dioxane, 1,3-dioxolane pyrrole, tetrahydropyran, and diazepan; wherein Het1 is optionally mono- or di-substituted on a carbon or a nitrogen with R₆; optionally mono- or di-substituted on a carbon with hydroxy, —N(R₆)₂, or —OR₆; optionally mono or di-substituted on a carbon with the mono-valent radicals —(C(R₆)₂)ₛOR₆ or —[(C(R₆)₂)ₛN(R₆)₂]; or optionally mono or di-substituted on a saturated carbon with divalent radicals ═O or —O(C(R₆)₂)ₛO—;
Het2 is a heteroaryl selected from the group comprising morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane pyrrole, and tetrahydropyran; wherein Het2 is optionally mono- or di-substituted on a carbon or a nitrogen with R₆; optionally mono- or di-substituted on a carbon with hydroxy, —N(R₆)₂, or —OR₆; optionally mono or di-substituted on a carbon with the mono-valent radicals —(C(R₆)₂)ₛOR₆ or —[(C(R₆)₂)ₛN(R₆)₂]; or optionally mono or di-substituted on a saturated carbon with divalent radicals ═O or —O(C(R₆)₂)ₛO—;
R₆ is hydrogen, an alkyl of 1-6 carbon atoms, an alkenyl of 2-6 carbon atoms, an alkynyl of 2-6 carbon atoms, a cycloalkyl of 3-6 carbon atoms, an alkanoyl of 2-7 carbon atoms, a carbamoylalkyl of 2-7 carbon atoms, a hydroxyalkyl of 1-6 carbon atoms, a hydroxycycloalkyl of 3-6 carbon atoms, a carboxyalkyl of 2-7 carbon atoms, pyrrolidine, piperidine, or imidazole optionally substituted with methyl;
a phenyl optionally mono-, di-, or tri-substituted with halogen, an alkoxy of 1-6 carbon atoms, trifluoromethyl, amino, an alkylamino of 1-3 carbon atoms, a dialkylamino of 2-6 carbon atoms, nitro, cyano, azido, halomethyl, an alkoxymethyl of 2-7 carbon atoms, an alkanoyloxymethyl of 2-7 carbon atoms, an alkylthio of 1-6 carbon atoms, hydroxy, a carboxyl, a alkoxycarbonyl of 2-7 carbon atoms, phenoxy, phenyl, thiophenoxy, benzoyl, benzyl, phenylamino, benzylamino, an alkanoylamino of 1-6 carbon atoms or alkyl of 1-6 carbon atoms;
Y is O, S, —NR₆C(O)—, —C(O)NR₆—, NR₆ or a bond;
Z¹ is O, S, —NR₆C(O)—, —C(O)NR₆—, NR₆ or a bond;
p is 0-4;
q is 0-4;
r is 0-4; and
s is 1-6.

31. The compound of claim 30, wherein R¹⁰ is t-butyl.

32. The compound of claim 30, wherein G₁, R₁, and R₄ are each, independently, hydrogen, halogen, alkyl of 1-6 carbon atoms, hydroxymethyl, halomethyl, alkoxy of 1-6 carbon atoms, alkylthio of 1-6 carbon atoms, trifluoromethyl, trifluoromethoxy, cyano, nitro, phenoxy, phenyl, thiophenoxy, benzyl, alkylamino of 1-6 carbon atoms, or dialkylamino of 2 to 12 carbon atoms.

33. The compound of claim 32, wherein G₁, R₁, and R₄ are each, independently, hydrogen, alkyl of 1-6 carbon atoms, trifluoromethyl, trifluoromethoxy or alkoxy of 1-6 carbon atoms.

34. The compound of claim 33, wherein R₁ is hydrogen.

35. The compound of claim 33, wherein R₄ is hydrogen.

36. The compound of claim 33, wherein G₁ is alkoxy of 1-6 carbon atoms.

37. The compound of claim 36, wherein G₁ is methoxy.

38. The compound of claim 30, wherein PG is benzyl.

39. A radiolabeled compound of formula (IIb): 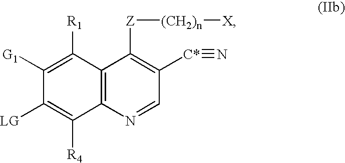
wherein:
X is a cycloalkyl of 3 to 7 carbon atoms, which may be optionally substituted with one or more alkyl of 1 to 6 carbon atoms, a pyridinyl, a pyrimidinyl, or a phenyl ring; wherein the pyridinyl, pyrimidinyl, or phenyl ring may be optionally mono-, di-, or tri-substituted with substituents selected from a group consisting of a halogen, an alkyl of 1-6 carbon atoms, an alkenyl of 2-6 carbon atoms, an alkynyl of 2-6 carbon atoms, azido, a hydroxyalkyl of 1-6 carbon atoms, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a benzoyl, an amino, an alkylamino of 1-6 carbon atoms, a dialkylamino of 2 to 12 carbon atoms, an alkanoylamino of 1-6 carbon atoms, an alkenoylamino of 3-8 carbon atoms, an alkynoylamino of 3-8 carbon atoms, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, a N,N-dialkylaminoalkyl of 3-10 carbon atoms, a N-alkylaminoalkoxy of 3-9 carbon atoms, a N,N-dialkylaminoalkoxy of 4-10 carbon atoms, mercapto, methylmercapto and benzoylamino;
a bicyclic aryl or bicyclic heteroaryl ring system of 8 to 12 atoms where the bicyclic heteroaryl ring contains 1 to 4 heteroatoms selected from N, O, and S, wherein the bicyclic aryl or bicyclic heteroaryl ring may be optionally mono- di-, tri, or tetra-substituted with a substituent comprising a halogen, an oxo, a thiocarbonyl, an alkyl of 1-6 carbon atoms, an alkenyl of 2-6 carbon atoms, an alkynyl of 2-6 carbon atoms, azido, a hydroxyalkyl of 1-6 carbon atoms, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, a N,N-dialkylaminoalkyl of 3-10 carbon atoms, an N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, and a benzoylamino;
a radical of the form: 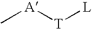
wherein;
A′ is a pyridinyl, a pyrimidinyl, or a phenyl ring; wherein the pyridinyl, pyrimidinyl, or phenyl ring may be optionally mono- or di-substituted with a substituent comprising an alkyl of 1-6 carbon atoms, an alkenyl of 2-6 carbon atoms, an alkynyl of 2-6 carbon atoms, azido, a hydroxyalkyl of 1-6 carbon atoms, a halogen, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, a N-alkylaminoalkyl of 2-9 carbon atoms, a N,N-dialkylaminoalkyl of 3-10 carbon atoms, an N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, a alkenoyloxy of 3-8 carbon atoms, an alkynoyloxy of 3-8 carbon atoms, a carbamoyl, a N-alkylcarbamoyl of 2-7 carbon atoms, a N,N-dialkylcarbamoyl of 3-13 carbon atoms, and benzoylamino;
T is substituted at a carbon of the pyridinyl, pyrimidinyl, or phenyl ring with —NH(CH₂)ₘ—, —O(CH₂)ₘ—, —S(CH₂)ₘ—, —NR(CH₂)ₘ—, —(CH₂)ₘ—, —(CH₂)ₘNH—, —(CH₂)ₘO—, —(CH₂)ₘS—, —SO(CH₂)ₘ—, —SO₂(CH₂)ₘ—, —CO(CH₂)ₘ—, —(CH₂)ₘCO—, —(CH₂)ₘSO—, —(CH₂)ₘSO₂— or —(CH₂)ₘNR—;
L is an imidazole or a phenyl ring wherein the imidazole or phenyl ring are optionally substituted at a carbon or nitrogen with one, two, or three substituents comprising an alkyl of 1-6 carbon atoms, an alkenyl of 2-6 carbon atoms, an alkynyl of 2-6 carbon atoms, azido, a hydroxyalkyl of 1-6 carbon atoms, a halogen, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, an N,N-dialkylaminoalkyl of 3-10 carbon atoms, a N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, a benzoylamino, a 5- or 6-membered heteroaryl ring where the heteroaryl ring contains 1 to 3 heteroatoms selected from N, O, and S and where the heteroaryl ring may be optionally mono- or di-substituted with a substituent comprising a halogen, an oxo, a thiocarbonyl, an alkyl of 1-6 carbon atoms, an alkenyl of 2-6 carbon atoms, an alkynyl of 2-6 carbon atoms, azido, a hydroxyalkyl of 1-6 carbon atoms, a halomethyl, an alkoxymethyl of 2-7 carbon atoms, an alkanoyloxymethyl of 2-7 carbon atoms, an alkoxy of 1-6 carbon atoms, an alkylthio of 1-6 carbon atoms, a hydroxy, a trifluoromethyl, a cyano, a nitro, a carboxy, an alkoxycarbonyl of 2-7 carbon atoms, an alkanoyl of 2-7 carbon atoms, a phenoxy, a phenyl, a thiophenoxy, a benzoyl, a benzyl, an amino, an alkylamino of 1-6 carbon atoms, a dialkylamino of 2 to 12 carbon atoms, a phenylamino, a benzylamino, an alkanoylamino of 1-6 carbon atoms, an alkenoylamino of 3-8 carbon atoms, an alkynoylamino of 3-8 carbon atoms, a carboxyalkyl of 2-7 carbon atoms, a carboalkoxyalkyl of 3-8 carbon atoms, an aminoalkyl of 1-5 carbon atoms, an N-alkylaminoalkyl of 2-9 carbon atoms, a N,N-dialkylaminoalkyl of 3-carbon atoms, a N-alkylaminoalkoxy of 3-9 carbon atoms, a N,N-dialkylaminoalkoxy of 4-10 carbon atoms, a mercapto, a methylmercapto, an alkanoyloxy of 1-6 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, and a benzoylamino;
LG is selected from halo, —O-triflate and —O-mesylate;
Z is —NH—, —O—, —S—, or —NR—,
R is alkyl of 1-6 carbon atoms,
G₁, R₁, and R₄ are each, independently, a hydrogen, a halogen, a hydroxy, an amino, a hydroxyamino, a trifluoromethyl, a trifluoromethoxy, a mercapto, an alkyl of 1-6 carbon atoms, a cycloalkyl of 3-8 carbon atoms, an alkenyl of 2-6 carbon atoms, an alkynyl of 2-6 carbon atoms, an alkenyloxy of 2-6 carbon atoms, an alkynyloxy of 2-6 carbon atoms, a hydroxyalkyl of 1-6 carbon, atoms, a mercaptoalkyl of 1-6 carbon atoms, a halomethyl, an alkoxymethyl of 2-7 carbon atoms, an alkoxy of 1-6 carbon atoms, a 2-methoxyethoxy, a 2-(2-methoxyethoxy)ethoxy, a cycloalkoxy of 3-8 carbon atoms, an alkylthio of 1-6 carbon atoms, a cycloalkylthio of 3-8 carbon atoms, an alkylsulfinyl of 1-6 carbon atoms, an alkylsulfonyl of 1-6 carbon atoms, an alkylsulfonamido of 1-6 carbon atoms, an alkenylsulfonamido of 2-6 carbon atoms, an alkynylsulfonamido of 2-6 carbon atoms, an alkylcarboxamido of 2-7 carbon atoms, a (N-alkyl)alkylcarboxamido of 3-13 carbon atoms, an alkenylcarboxamido of 3-7 carbon atoms, an (N-alkyl)alkenylcarboxamido of 4-13 carbon atoms, an alkynylcarboxamido of 3-7 carbon atoms, an (N-alkyl)alkynylcarboxamido of 4-13 carbon atoms, cyano, nitro, carboxy, a alkoxycarbonyl of 2-7 carbon atoms, an alkanoyl of 2-7 carbon atoms, an alkenoyl of 3-7 carbon atoms, a N-alkyl-N-alkenylamino of 4 to 12 carbon atoms, an N,N-dialkenylamino of 6-12 carbon atoms, phenylamino, benzylamino, phenoxy, phenyl, thiophenoxy, benzyl, an alkylamino of 1-6 carbon atoms, an alkanoyloxy of 2-7 carbon atoms, an alkenoyloxy of 3-8 carbon atoms, an alkynoyloxy of 3-8 carbon atoms, a carbamoyl, an N-alkylcarbamoyl of 2-7 carbon atoms, a N,N-dialkylcarbamoyl of 3-13 carbon atoms, a dialkylamino of 2 to 12 carbon atoms, an alkanoyloxymethyl group of 2-7 carbon atoms, an alkenoyloxymethyl group of 2-7 carbon atoms, an alkynoyloxymethyl group of 2-7 carbon atoms, azido, benzoyl, a carboxyalkyl of 2-7 carbons, and a carboalkoxyalkyl of 3-8 carbon atoms,
—NR₆C(O)H; —N(C(O)R₆)C(O)R₆;
—Y—(C(R₆)₂)ₚ-Het1;
—Y—(C(R₆)₂)ₚ-Het1-(C(R₆)₂)_{q}-Z¹-(C(R₆)₂)ᵣ-Het2;
Het1 is a 3-8 membered saturated heterocyclic ring containing one or more nitrogen, oxygen or sulfur atoms such as, but not limited to morpholine, thiomorpholine, thiomorpholine S-oxide, thiomorpholine S,S-dioxide, piperidine, pyrrolidine, aziridine, piperazine, tetrahydrothiophene, tetrahydrofuran, dioxane, 1,3-dioxolane pyrrole, tetrahydropyran, and diazepan; wherein Het1 is optionally mono- or di-substituted on a carbon or a nitrogen with R₆; optionally mono- or di-substituted on a carbon with hydroxy, —N(R₆)₂, or —OR₆; optionally mono or di-substituted on a carbon with the mono-valent radicals —(C(R₆)₂)ₛOR₆ or —[(C(R₆)₂)ₛN(R₆)₂]; or optionally mono or di-substituted on a saturated carbon with divalent radicals ═O or —O(C(R₆)₂)ₛO—;
Het2 is a heteroaryl selected from the group comprising morpholine, thiomorpholine, thiomorpholine S-oxide, thiomorpholine S,S-dioxide, piperidine, pyrrolidine, aziridine, pyridine, imidazole, 1,2,3-triazole, 1,2,4-triazole, thiazole, thiazolidine, tetrazole, piperazine, furan, thiophene, tetrahydrothiophene, tetrahydrofuran, dioxane, 1,3-dioxolane pyrrole, and tetrahydropyran; wherein Het2 is optionally mono- or di-substituted on a carbon or a nitrogen with R₆; optionally mono- or di-substituted on a carbon with hydroxy, —N(R₆)₂, or —OR₆; optionally mono or di-substituted on a carbon with the mono-valent radicals —(C(R₆)₂)ₛOR₆ or —[(C(R₆)₂)ₛN(R₆)₂]; or optionally mono or di-substituted on a saturated carbon with divalent radicals ═O or —O(C(R₆)₂)ₛO—;
R₆ is hydrogen, an alkyl of 1-6 carbon atoms, an alkenyl of 2-6 carbon atoms, an alkynyl of 2-6 carbon atoms, a cycloalkyl of 3-6 carbon atoms, an alkanoyl of 2-7 carbon atoms, a carbamoylalkyl of 2-7 carbon atoms, a hydroxyalkyl of 1-6 carbon atoms, a hydroxycycloalkyl of 3-6 carbon atoms, a carboxyalkyl of 2-7 carbon atoms, pyrrolidine, piperidine, or imidazole optionally substituted with methyl;
a phenyl optionally mono-, di-, or tri-substituted with halogen, an alkoxy of 1-6 carbon atoms, trifluoromethyl, amino, an alkylamino of 1-3 carbon atoms, a dialkylamino of 2-6 carbon atoms, nitro, cyano, azido, halomethyl, an alkoxymethyl of 2-7 carbon atoms, an alkanoyloxymethyl of 2-7 carbon atoms, an alkylthio of 1-6 carbon atoms, hydroxy, a carboxyl, a alkoxycarbonyl of 2-7 carbon atoms, phenoxy, phenyl, thiophenoxy, benzoyl, benzyl, phenylamino, benzylamino, an alkanoylamino of 1-6 carbon atoms or alkyl of 1-6 carbon atoms;
Y is O, S, —NR₆C(O)—, —C(O)NR₆—, NR₆ or a bond;
Z¹ is O, S, —NR₆C(O)—, —C(O)NR₆—, NR₆ or a bond;
p is 0-4;
q is 0-4;
r is 0-4; and
s is 1-6.
